(19) United States
(12) Patent Application Publication
(10) Pub. No.: US 2023/0248891 A1
(43) Pub. Date: Aug. 10, 2023

(54) SYSTEMS AND METHODS FOR AUTOMATIC BIDIRECTIONAL PRIMING OF A GAS-ENRICHMENT SYSTEM

(71) Applicant: Zoll Circulation, Inc., San Jose, CA (US)

(72) Inventors: Stephen Elwood Myrick, Tustin, CA (US); Christo Petrov Pamichev, Cupertino, CA (US); Gary A. Freeman, Waltham, MA (US)

(21) Appl. No.: 18/104,750

(22) Filed: Feb. 1, 2023

Related U.S. Application Data
(60) Provisional application No. 63/305,952, filed on Feb. 2, 2022.

(51) Int. Cl. A61M 1/36

(52) U.S. Cl. CPC A61M 1/3621; A61M 2205/3331; A61M 1/3643

(57) ABSTRACT

Methods and systems for bidirectional priming of a blood circuit while a catheter is connected to the circuit that delivers gas-enriched blood to a patient. The system primes the circuit while the catheter is connected to the circuit by controlling a first flow control mechanism to close to prevent blood flow through the draw line to a catheter and causes a pump to circulate blood in a first direction through a mixing chamber and/or through a bubble trap that removes air bubbles from the circuit. The system controls a second flow control mechanism to close to prevent blood flow in a return line to the catheter while causing the first flow control mechanism to open after the second flow control mechanism is closed and while causing the pump to circulate the blood in a second, opposite direction through the mixing chamber that removes air bubbles from the circuit.

### CLAIM OF PRIORITY

This application claims priority under 35 U.S.C. § 119(e) to U.S. Patent Application Ser. No. 63/305,952, filed on Feb. 2, 2022, the entire contents of which are hereby incorporated by reference.

### TECHNICAL FIELD

The disclosure relates to systems and methods for the delivery of gas-enriched blood into a patient.

### BACKGROUND

Gas-enriched liquids are desirable in a wide variety of applications. However, at ambient pressure, the relatively low solubility of many gases, such as oxygen or nitrogen, within a liquid, such as water, produces a relatively low concentration of the dissolved gas in the liquid. One method of obtaining an increase in the gas concentration level without significant increase in liquid volume involves an injection and mixing of a gas-enriched liquid, such as a gas-supersaturated liquid, into a liquid of interest. A liquid can be gas-enriched at high pressure.

Conventional methods for the delivery of oxygenated blood or oxygen-enriched liquids to tissues and bodily liquids involve the use of extracorporeal circuits for blood oxygenation. Extracorporeal circuits require withdrawing blood from a patient, circulating the blood through an oxygenator to increase blood oxygen concentration, and then delivering the blood back to the patient.

### SUMMARY

This document describes a gas-enrichment system configured to deliver gas-enriched blood intravenously to a patient. The system for delivering gas-enriched blood within the vasculature of a patient (hereinafter the delivery system) is configured to connect to a catheter device to deliver the gas-enriched blood to the patient. The delivery system includes a blood circuit having a draw line and a return line. The draw line and return line are configured to connect to the catheter. Blood is withdrawn from the patient via the draw line. The blood is mixed with a gas-enriched liquid, or oxygen-enriched liquid such as a supersaturated oxygen (SSO₂) enriched liquid, to create gas-enriched blood. The gas-enriched blood is delivered back to the patient through the catheter via the return line.

Before the delivery system delivers gas-enriched blood to the patient, the blood circuit is primed with blood. Priming the blood circuit removes or displaces air bubbles and/or room air from the unprimed blood circuit and fills the blood circuit with blood. In certain implementations, removal or venting of room air and/or air bubbles from the blood circuit may refer to removal or venting of air or atmospheric air or ambient air that is present in the blood circuit, and not removal or venting of gasses that are dissolved in gas-enriched blood present in the blood circuit. Room air and/or air bubbles may include atmosphere, ambient or other air that is present in an environment in which the blood circuit is present. Room air including atmosphere, ambient or other air that is in the environment of the gas-enrichment system and/or air bubbles may be present within the blood circuit prior to the priming process. The priming process removes this room air and/or air bubbles from the blood circuit. Priming the blood circuit prevents any room air or air bubbles from entering the vasculature of the patient, which could result in embolism in the patient.

For gas-enrichment therapy, the catheter is inserted into a vasculature of a patient. The catheter is configured for delivering gas-enriched blood within the vasculature of a patient. The catheter is connected to a mixing chamber of the delivery system, which is configured to mix the blood of the patient with a gas-enriched liquid. The catheter includes one or more lumens configured to receive the gas-enriched blood from the mixing chamber. In some implementations, the catheter may include one or more occlusion structures configured to partially obstruct a flow of blood within the vasculature of the patient while allowing delivery of the gas-enriched blood to the region of the vasculature.

The delivery system and priming processes described in this specification may enable one or more of the following advantages. Certain existing systems that provide gas-enriched blood to a patient require a blood circuit and components of the blood circuit to be primed with blood to remove the room air and/or air bubbles from a blood circuit while the return line is disconnected from the catheter. In these systems, room air and/or air bubbles are pushed out of a return line (return tubing) by operating a pump while the return line is disconnected from the catheter (e.g., an open loop priming). The pump operates to fill the pump tubing and a blood mixing chamber with blood and remove room air and/or air bubbles from the blood circuit. However, blood is also expelled from the disconnected return line during priming. This results in a connection that is manually performed and which may introduce air bubbles into the circuit if connected too soon or blood loss if connection is delayed. Additionally, a user of such systems is occupied by the priming process and must monitor the system to determine when to connect the catheter and complete the priming process. Additionally, multiple operators may be needed to prime the delivery system and perform the “wet-to-wet” connection (also known as wet connection, meaning the connection of a fluid-primed return line to a fluid-primed catheter) of the overflowing return line to the intravenous catheter. In such systems, due to the need to mitigate the possibility that the return line is connected too soon or too late, a priming button must be pressed and held throughout the duration of the priming, requiring a first operator to hold the priming button while witnessing the wet connection and a second operator to perform the wet connection.

The delivery systems described herein overcome the above described challenges by enabling the blood circuit to be primed after the catheter is connected to the blood circuit (e.g., a closed loop priming). A single press and release of a priming button (a one-touch activation) can initiate the priming process. The delivery system is configured to provide automatic, bidirectional priming of the blood circuit and remove all room air and/or air bubbles from the blood circuit without additional intervention from a user. A single user can operate the delivery system to perform the entire priming process. The closed loop, bidirectional priming thus reduces chances for manual error. The automated priming process is safer because there is less opportunity to accidentally introduce air bubbles to the blood circuit from connecting too soon and less opportunity for blood loss due to delayed connection. There is a reduced or eliminated possibility of introducing contamination in the blood circuit during priming. Contamination may occur when the disconnected return line is primed and connected to the catheter in a wet-connect procedure.

To prime each of the draw line (draw tubing) and the return line, a pump of the delivery system is configured for bidirectional operation during the priming process, also called a bidirectional priming process. The bidirectional priming process enables the blood circuit to be primed while the catheter is connected to the delivery system. This eliminates a “wet to wet” connection step of the return line to the catheter. Additionally, the delivery system is configured to automatically complete the entire priming process without requiring a user to adjust air or bubble traps, activate the blood pump, or otherwise interact with the delivery system when the priming process is performed.

To perform the bidirectional priming, the catheter, after being inserted into the vasculature of the patient, is connected to the delivery system. Connecting the catheter to the delivery system includes connecting a draw line from the catheter and a return line to the catheter. The draw and return lines are configured to be in fluid communication with a plurality of chambers of the delivery system. While the catheter is connected, to prime the blood circuit, the delivery system operates a pump of the blood circuit in a first direction to prime the return line of the catheter. The draw line from the catheter is closed during operation of the pump in the first direction. Once the return line is primed with blood and room air and/or air bubbles are removed from the blood circuit, the draw line is reopened. The return line is then closed, and the delivery system reverses the pump to operate in a second direction, pumping blood in the direction of the closed return tubing. The room air and/or air bubbles are removed from the draw line to prime the draw line. Once each of the draw line and the return line are primed, the lines are opened and the delivery system is ready to perform delivery of gas-enriched blood to the patient. A flow control mechanism configured to control (e.g., by blocking or restricting) the passage of blood through the blood circuit and through the draw and return lines may be used to open and close the draw and return lines. Such a mechanism may include a clamp, valve, gate, flow regulator, or other similar mechanism that enables flow of the blood to be controlled (e.g., stopped).

As previously stated, each of the priming operations can be performed automatically (without user intervention). If a fault is detected, such as air bubbles being trapped in the blood circuit, a flow control mechanism failing, or any such issue with the delivery system, a safety shut-off can automatically occur. For example, if bubbles are detected in either the draw line or the return line, the delivery system is configured to cease delivery of gas-enriched fluid to the patient and generate an alarm or alert. In some implementations, the delivery system is configured to display, by a user interface, operational data of the priming operations to inform a user as to the status of the delivery system during priming.

The following aspects enable one or more of the previously described advantages.

In a general aspect, a system is for delivering gas-enriched blood within a vasculature of a patient, the system configured for priming of a blood circuit of the system. The system includes a blood circuit. The blood circuit includes a pump configured to circulate blood in the blood circuit, a mixing chamber configured to mix blood of the patient with a gas-enriched liquid to form a gas-enriched blood, a draw line coupled to the mixing chamber and configured to connect a catheter to the mixing chamber, a return line coupled to the mixing chamber and configured to connect the catheter to the mixing chamber, a first vent and a second vent, wherein each vent is configured to vent room air and/or air bubbles from the blood circuit, a first flow control mechanism configured to control blood flow in the draw line to the catheter, and a second flow control mechanism configured to control blood flow in the return line to the catheter. The system includes a controller configured to provide bidirectional priming of the blood circuit by controlling operation of the pump, the first flow control mechanism and the second flow control mechanism to perform operations. The operations include causing the blood to flow in a first direction through the blood circuit wherein room air and/or air bubbles that are present in the blood circuit are removed from the blood circuit through the first vent to prime the return line, and causing the blood to flow in a second, opposite direction through the blood circuit wherein room air and/or air bubbles that are present in the blood circuit are removed from the blood circuit through the second vent to prime the draw line, wherein the bidirectional priming of the blood circuit allows for priming of the blood circuit while the draw and return lines are connected to the catheter.

In some implementations, the blood circuit further comprises a bubble trap, which may be positioned on a first side of the blood pump opposite the mixing chamber. The mixing chamber may be positioned on a second side of the blood pump.

In some implementations, the first vent is coupled to the bubble trap. The second vent may be coupled to the mixing chamber. The vents may be coupled such that the room air and/or air bubbles that are present in the blood circuit exit the blood circuit through the first vent, via the bubble trap, and through the second vent, via the mixing chamber.

In some implementations, the gas-enriched liquid is oxygen-enriched liquid, which may have a dissolved O₂ concentration of 0.1-6 ml O₂/ml liquid.

In some implementations, the gas-enriched blood is oxygen-enriched blood, which may have an elevated pO₂ of 600-1500 mmHg.

In some implementations, the system includes a catheter coupled to the mixing chamber. The catheter may be configured to be inserted into a vasculature of a patient and may deliver gas-enriched blood to a region of the vasculature of the patient. The catheter may comprise one or more lumens, which may be configured to receive the gas-enriched blood from the mixing chamber.

In some implementations, the data processing system includes a pump tube. The pump tube may be configured to interface with the pump. The pump tube may be compressed by the pump to circulate the blood in the blood circuit. The pump may be configured to pump the blood in both the first direction in the blood circuit and the second direction in the blood circuit. The data processing system may include a first pressure sensor on the pump tube. The first pressure sensor may be configured to measure a first pressure in the pump tube on a first side of the pump. In some implementations, the data processing system may include a second pressure sensor on the pump tube. The second pressure sensor may be configured to measure a second pressure in the pump tube on a second side of the pump opposite the first side. In some implementations, bidirectional priming occurs with a single pressure sensor, whether there is one vent or a plurality of vents in the blood circuit.

In some implementations, the operations include causing the first flow control mechanism to close. The operations may include causing the second flow control mechanism to open. The operations may include causing the pump to circulate the blood in the first direction of the blood circuit towards the closed first flow control mechanism until the first pressure measured by the first pressure sensor exceeds a first threshold pressure value. The operations may include causing the pump to circulate the blood in the blood circuit in the second direction. The operations may include, in response to the first pressure measured by the first pressure sensor exceeding the first threshold pressure value, causing the pump to circulate the blood in the blood circuit in the second direction.

In some implementations, the operations include causing the second flow control mechanism to close. The operations may include causing the first flow control mechanism to open. The operations may include causing the pump to circulate the blood in the blood circuit in the second direction. The operations may include causing the pump to circulate the blood in the blood circuit in the second direction towards the closed second flow control mechanism until the second pressure measured by the second pressure sensor exceeds a second threshold pressure value. The operations may include determining that priming is completed. The operations may include, in response to the second pressure exceeding the second threshold pressure value, determining that priming is completed.

In some implementations, the operations include, in response to determining that priming is completed, causing both the first flow control mechanism and the second flow control mechanism to open. The operations may include causing the pump to circulate blood through a catheter coupled to the draw line and the return line.

In some implementations, the data processing system includes a pump tube. The pump tube may be configured to interface with the pump. The pump tube may be a part of the blood circuit. The pump tube may connect the mixing chamber and a bubble trap that may comprise the vent.

In some implementations, the operations include performing data logging of sensor data from one or more of a pressure sensor, a flow sensor, a blood level sensor, a valve, the first flow control mechanism, the second flow control mechanism, and the pump. The operations may include storing the sensor data in a data store.

In some implementations, the data store comprises a local data store.

In some implementations, the data store comprises a remote data store.

In some implementations, the remote data store comprises cloud storage.

In some implementations, performing data logging comprises structuring the sensor data to enable querying of the sensor data by a remote computing system.

In some implementations, performing data logging comprises: generating a data entry for a priming process. Performing data logging may comprise storing the data entry. The data entry may comprise a start time when priming is initiated. The data entry may comprise a stop time when priming is completed. The data entry may comprise the sensor data associated with the priming process.

In some implementations, performing data logging comprises generating a first data item indicating when the priming process is in a first phase. The first phase may represent when the controller is causing the blood to flow in the first direction through the blood circuit through the mixing chamber to prime the return line. Performing data logging may comprise generating a second data item indicating when the priming process is in a second phase. The second phase may represent when the controller is causing the blood to flow in the second, opposite direction through the blood circuit to prime the draw line.

In some implementations, the data entry comprises the first data item and the second data item. The data entry may associate sensor data generated during the first phase with the first data item. The data entry may associate sensor data generated during the second phase with the second data item.

In some implementations, the operations include receiving sensor data from one or more of a pressure sensor, blood level sensor, flow sensor, the pump, the first flow control mechanism, and the second flow control mechanism. The operations may include determining, based on the sensor data, that a priming process is successful or unsuccessful.

In some implementations, determining, based on the sensor data, whether the priming process is successful or unsuccessful may comprises comparing pressure data to a threshold and may comprise, optionally in response to determining that the pressure data satisfies the threshold, determining that the priming process is successful.

In some implementations, determining, based on the sensor data, that the priming process is successful or unsuccessful comprises receiving, from a first blood level sensor in the mixing chamber, first blood level data indicating that the mixing chamber is full of blood. In some implementations, determining, based on the sensor data, that the priming process is successful or unsuccessful comprises receiving, from a second blood level sensor in a bubble trap, second blood level data indicating that the bubble trap is full of blood. Determining, based on the sensor data, that the priming process is successful or unsuccessful may comprise, in response to receiving the first blood level data and the second blood level data, determining that the priming process is successful.

In some implementations, the system includes a control. The control may be configured to initiate operation of the controller to control operation of one or more of the pump, the first flow control mechanism, and the second flow control mechanism for causing the blood to flow in the first direction through the blood circuit through the mixing chamber to prime the return line and/or causing the blood to flow in the second, opposite direction through the blood circuit to prime the draw line.

In some implementations, the control is a button. Operation of the controller may be initiated in response to a single press and release of the button. The button may be a physical button or virtual button on a user interface.

In some implementations, upon insertion of a cartridge comprising the mixing chamber into a cartridge housing of a console housing supporting the blood circuit, the draw line from the cartridge automatically self-aligns with the draw flow control mechanism positioned on the console, and/or the return line automatically aligns with the return flow control mechanism positioned on the console.

In some implementations, the data processing system includes a bubble detector. The bubble detector may be configured to generate a signal indicative of a presence of an air bubble in the blood circuit. The controller may be configured to perform operations comprising one or more of: during delivery of the gas-enriched blood within the vasculature of the patient, receiving, from the bubble detector, the signal indicative of the presence of the air bubble in the blood circuit; in response to receiving the signal, causing either the first flow control mechanism or the second flow control mechanism to close and pausing the delivery of the gas-enriched blood within the vasculature of the patient; re-priming the blood circuit to remove the air bubble from the blood circuit; and restarting the delivery of the gas-enriched blood within the vasculature of the patient.

In some implementations, each of the first flow control mechanism and the second flow control mechanism comprises one or more of a clamp, valve, or flow regulator.

In a general aspect, a cartridge is for interfacing with a console configured for delivering gas-enriched blood within a vasculature of a patient. The cartridge may be configured for priming of a blood circuit of the delivery system. The cartridge may include one or more of a fluid supply chamber configured to draw liquid from an external liquid source; a gas-enrichment chamber configured to receive a source of gas for enriching the liquid from the external liquid source to provide a gas-enriched liquid; a mixing chamber configured to mix the gas-enriched liquid with blood from a patient; and a bubble trap configured to remove room air and/or air bubbles from a blood circuit that is configured to be connected to the mixing chamber and an external intravenous catheter. The bubble trap may have a vent for removing room air and/or air bubbles from the blood circuit during a bi-directional priming process.

In some implementations, the bubble trap comprises a solenoid. The bubble trap or solenoid may be configured to receive a control signal to control operation of the vent for removing room air and/or air bubbles form the blood circuit.

In some implementations, the bubble trap is oriented for placement between a draw clamp and a blood pump of the console when the cartridge is interfaced with the console. The bubble trap may be configured to vent room air and/or air bubbles when the draw clamp is closed.

In some implementations, the mixing chamber includes a solenoid. The mixing chamber or solenoid may be configured to receive a control signal to control operation of the vent for removing room air and/or air bubbles form the blood circuit.

In some implementations, the mixing chamber is oriented for placement between a return clamp and a blood pump of the console when the cartridge is interfaced with the console. The mixing chamber may be configured to vent room air and/or air bubbles when the return clamp is closed.

In some implementations, the fluid supply chamber comprises a piston and optionally a piston actuator. The piston actuator may be configured to drive the piston to draw liquid from the external liquid source.

In some implementations, the cartridge includes a first temperature sensor. The first temperature sensor may be configured for measuring a first temperature of blood in a draw line. The cartridge may include a second temperature sensor. The second temperature sensor may be configured for measuring a second temperature of blood in a return line.

In some implementations, the cartridge includes a first receptacle. The first receptacle may be configured for receiving a pump to enable the pump to interface with the blood circuit. The cartridge may include a second receptacle. The second receptacle may be configured for receiving a draw clamp. The draw clamp may be configured to control blood flow through a draw line of the cartridge. The cartridge may include a third receptacle. The third receptacle may be configured for receiving a return clamp. The return clamp may be configured to control the blood flow through a return line of the cartridge.

In some implementations, the cartridge includes a seal. The seal may be configured to engage a housing of the console. The seal may be configured to receive pressure when the cartridge is operating for delivery of gas-enriched blood. The seal may prevent room air and/or air bubbles from entering the cartridge.

In some implementations, the seal comprises an O-ring. In some implementations, the seal is coupled to the bubble trap.

In some implementations, the cartridge includes a draw line configured to connect to the external intravenous catheter; and a return line configured to connect to the external intravenous catheter; wherein the draw line is oriented to interface with a first clamp that restricts blood flow in the draw line; and wherein the return line is oriented to interface with a second clamp that restricts blood flow in the return line.

In some implementations, the cartridge includes a first pressure sensor. The first pressure sensor may be provided on a pump tube. The pump tube may be configured for interfacing with a pump. The first pressure sensor may be configured to measure a pressure in the pump tube on a first side of the pump. The cartridge may comprise a second pressure sensor. The second pressure sensor may be provided on the pump tube. The second pressure sensor may be configured to measure the pressure in the pump tube on a second side of the pump opposite the first side.

In some implementations, the cartridge includes a pump tube. The pump tube may be configured to interface with a pump of the console when the cartridge is interfaced with the console. The pump tube may be a part of the blood circuit. The pump tube may connect the mixing chamber and the bubble trap.

In some implementations, two or more of the fluid supply chamber, the gas-enrichment chamber, the mixing chamber and the bubble trap are in the same housing.

In some implementations, upon insertion of the cartridge into a cartridge housing of a console, tubing from the cartridge automatically self-aligns with one or more of a draw flow control mechanism, a return flow control mechanism and a pump.

In some implementations, each of the first flow control mechanism and the second flow control mechanism comprises one or more of a clamp, valve, or flow regulator.

In a general aspect, a delivery system for delivering gas-enriched blood within a vasculature of a patient, the delivery system configured for priming of a blood circuit of the delivery system. The delivery system includes a blood circuit, comprising a pump configured to circulate blood in the blood circuit, a mixing chamber configured to mix blood of the patient with a gas-enriched liquid, a draw line coupled to the mixing chamber and configured to connect a catheter to the mixing chamber and to interface with a first flow control mechanism, a return line coupled to the mixing chamber and configured to connect the catheter to the mixing chamber and to interface with a second flow control mechanism; and a controller configured to control operation of the pump and operation of the first and second flow control mechanisms to perform bidirectional priming of the blood circuit while the catheter is connected to the blood circuit, the controller configured for alternating a direction of blood flow through the blood circuit and alternating closure of the first and second flow control mechanisms to block blood flow in the draw line and return line and prevent room air and/or air bubbles from flowing to the catheter during priming.

In some implementations, the controller is configured to perform operations comprising closing the first flow control mechanism when causing the blood to flow in reverse direction, the first flow control mechanism blocking blood flow in the draw line.

In some implementations, the controller is configured to perform operations comprising closing the second flow control mechanism and opening the first flow control mechanism when causing the blood to flow in a forward direction, the second flow control mechanism blocking blood flow in the return line.

In some implementations, the controller is configured to perform operations comprising: measuring, by a first pressure sensor, a first pressure in the blood circuit between a pump and the first flow control mechanism in the blood circuit when the pump is causing the blood to flow in a first direction through the blood circuit. The controller is configured for comparing the first pressure to a threshold value. The controller is configured for determining that the return line is primed when the first pressure exceeds the threshold value.

In some implementations, the controller is configured to perform operations comprising: measuring, by a second pressure sensor, a second pressure in the blood circuit between a pump and a second flow control mechanism in the blood circuit when the pump is causing the blood to flow in a second direction through the blood circuit. The controller is configured for comparing the second pressure to a threshold value. The controller is configured for determining that the draw line is primed when the first pressure exceeds the threshold value.

In some implementations, the controller is configured to perform operations comprising: determining that the draw line is primed, determining that the return line is primed, and in response to determining each of the draw line and the return line are primed, causing circulation of blood through the catheter coupled to the draw line and the return line.

In some implementations, the controller is configured to perform operations comprising: receiving sensor data from one or more of a pressure sensor, blood level sensor, a pump, a first flow control mechanism, and a second flow control mechanism and determining, based on the sensor data, that a priming process is successful or unsuccessful. In some implementations, determining, based on the sensor data, that the priming process is successful or unsuccessful comprises: comparing pressure data to a threshold and in response to determining that the pressure data satisfies the threshold, determining that the priming process is successful.

In some implementations, determining, based on the sensor data, that the priming process is successful or unsuccessful comprises: receiving, from a first blood level sensor in the mixing chamber, first blood level data indicating that the mixing chamber is full of blood, receiving, from a second blood level sensor in a bubble trap second blood level data indicating that the bubble trap is full of blood, and in response to receiving the first blood level data and the second blood level data, determining that the priming process is successful.

In some implementations, the controller is configured to perform operations comprising: determining that a control is actuated; and in response to determining, automatically causing the blood to flow in a first direction through the blood circuit through the mixing chamber to prime the return line and automatically causing the blood to flow in a second, opposite direction through the blood circuit to prime the draw line.

In some implementations, the controller is configured to perform operations comprising: generating a data log comprising operational data that describes a bidirectional priming of the blood circuit. In some implementations, the controller is configured to perform operations comprising: detecting that the bidirectional priming of the blood circuit is completed and in response to detecting, sending the data log to a remote storage comprising cloud storage.

In some implementations, the controller is configured to perform operations comprising: receiving a query for data describing operation of a pump, a pressure sensor, a temperature sensor, a first flow control mechanism on the draw line, or a second flow control mechanism on the return line and in response to receiving the query, sending at least a portion of the data log to a remote device.

In some implementations, the controller is configured to perform operations comprising: determining that a value included in the data log is outside an expected range for that value; and generating data indicating that an error occurred during the bidirectional priming of the blood circuit.

In some implementations, the gas-enriched liquid is oxygen-enriched liquid, which may have a dissolved O₂ concentration of 0.1-6 ml O₂/ml liquid.

In some implementations, the gas-enriched blood is oxygen-enriched blood, which may have an elevated pO₂ of 600-1500 mmHg.

In a general aspect, a process is for bidirectional priming of a blood circuit while a catheter is connected to the blood circuit. The blood circuit is configured for delivering gas-enriched blood to a vasculature of a patient. The process includes providing a blood circuit comprising a mixing chamber configured to mix blood of the patient with a gas-enriched liquid to form the gas-enriched blood, a first vent, a second vent, a draw line, a return line, and a catheter, wherein the draw line and return line are connected to the catheter; and while the catheter is connected to the blood circuit: causing blood to flow in a first direction through the blood circuit through the mixing chamber wherein room air and/or air bubbles that are present in the blood circuit exits the blood circuit through the first vent to prime the return line; and causing the blood to flow in a second, opposite direction through the blood circuit through the mixing chamber wherein room air and/or air bubbles that are present in the blood circuit exits the blood circuit through the second vent to prime the draw line.

In some implementations, the process includes closing the first clamp when causing the blood to flow in the first direction, which may cause the first clamp to block blood flow in the draw line.

In some implementations, the process includes closing the second clamp and opening the first clamp when causing the blood to flow in the second direction, which may cause the second clamp to block blood flow in the return line.

In some implementations, the process includes one or more of measuring, by a first pressure sensor, a first pressure in the blood circuit between a pump and the first clamp in the blood circuit when the pump is causing the blood to flow in the first direction through the blood circuit; comparing the first pressure to a threshold value; and determining that the return line is primed when the first pressure exceeds the threshold value.

In some implementations, the process includes one or more of measuring, by a second pressure sensor, a second pressure in the blood circuit between a pump and the second clamp in the blood circuit when the pump is causing the blood to flow in the second direction through the blood circuit; comparing the second pressure to a threshold value; and determining that the draw line is primed when the first pressure exceeds the threshold value.

In some implementations, the process includes one or more of determining that the draw line is primed; determining that the return line is primed; and in response to determining each of the draw line and the return line are primed, causing circulation of blood through a catheter coupled to the draw line and the return line.

In some implementations, the process includes receiving sensor data from one or more of a pressure sensor, blood level sensor, a pump, a first clamp, and a second clamp. The process may include determining, based on the sensor data, that a priming process is successful or unsuccessful.

In some implementations, determining, based on the sensor data, whether the priming process is successful or unsuccessful comprises: comparing pressure data to a threshold; and may comprise, in response to determining that the pressure data satisfies the threshold, determining that the priming process is successful.

In some implementations, determining, based on the sensor data, that the priming process is successful or unsuccessful comprises one or more of: receiving, from a first blood level sensor in the mixing chamber, first blood level data indicating that the mixing chamber is full of blood; receiving, from a second blood level sensor in a bubble trap second blood level data indicating that the bubble trap is full of blood; and in response to receiving the first blood level data and the second blood level data, determining that the priming process is successful.

In some implementations, the process includes actuating a control; and, in response to actuation of the control, automatically causing the blood to flow in the first direction through the blood circuit through the mixing chamber to prime the return line and automatically causing the blood to flow in the second, opposite direction through the blood circuit to prime the draw line.

In some implementations, the process includes generating a data log. The data log may comprise operational data. The operational data may describe the bidirectional priming of the blood circuit.

In some implementations, the process includes detecting that the bidirectional priming of the blood circuit is completed, and may comprise, in response to detecting, sending the data log to a remote storage comprising cloud storage.

In some implementations, the process includes receiving a query for data. The query may describe operation of one or more of a pump, a pressure sensor, a temperature sensor, a first clamp on the draw line, and a second clamp on the return line. The process may comprise, in response to receiving the query, sending at least a portion of the data log to a remote device.

In some implementations, the process includes determining that a value included in the data log is outside an expected range for that value; and may comprise, optionally based on the value, generating data or an indication indicating that an error occurred during the bidirectional priming of the blood circuit.

In a general aspect, a process is for bidirectional priming of a blood circuit while a catheter is connected to the blood circuit. The blood circuit is configured for delivering gas-enriched blood to a vasculature of a patient. The process includes providing a blood circuit comprising a mixing chamber configured to mix blood of the patient with a gas-enriched liquid to form the gas-enriched blood, a draw line, a return line, and a catheter, wherein the draw line and return line are connected to the catheter; and while the catheter is connected to the blood circuit: controlling a first flow control mechanism to close to prevent blood flow through the draw line to a catheter; causing a pump to circulate blood in a first direction through the mixing chamber and through a bubble trap configured to remove room air and/or air bubbles from the blood circuit, the first direction being in a direction in the blood circuit toward the first flow control mechanism from the pump, the first flow control mechanism being closed; controlling a second flow control mechanism to close to prevent blood flow in a return line to the catheter; controlling the first flow control mechanism to open after the second flow control mechanism is closed; and causing the pump to circulate the blood in a second direction through the mixing chamber configured to remove room air and/or air bubbles from the blood circuit, the second direction being opposite the first direction, the second direction being in a direction in the blood circuit toward the second flow control mechanism from the pump, the second flow control mechanism being closed and the first flow control mechanism being open.

In some implementations, each of the first flow control mechanism and the second flow control mechanism comprises one or more of a clamp, valve, or flow regulator.

In a general aspect, a method for delivering gas-enriched blood within a vasculature of a patient is disclosed for priming of a blood circuit of a delivery system. The method comprises: activating a pump in a forward direction to circulate blood in the blood circuit to move blood up a draw line from a catheter in the vasculature of a patient toward a blood mixing chamber, a clamp being closed on a return line to increase a pressure at a blood mixing chamber; venting air from the blood circuit to lower the pressure at the blood mixing chamber; activating the pump in a reverse direction to decrease a pressure in a return line of the blood circuit; opening a return clamp to enable blood to move up the return line to the blood mixing chamber and prime the return line; activating the pump in the reverse direction to move blood up the return line; and venting the air from the return line to prime the return line.

In some implementations, the blood is mixed with a gas-enriched liquid comprising oxygen-enriched liquid, which may have a dissolved O₂ concentration of 0.1-6 ml O₂/ml liquid to form a gas-enriched blood.

In some implementations, the gas-enriched blood is oxygen-enriched blood, which may have an elevated pO₂ of 600-1500 mmHg.

In some implementations, the method further comprises performing a test of one or more of a clamp, flow sensor, pressure sensor, or pump. The method may comprise receiving one or more signals indicative of a fault in operation of one or more of the clamp, the flow sensor, the pressure sensor, or the pump. The method may comprise, responsive to receiving the one or more signals, preventing operation of the pump.

In some implementations, the method further comprises determining that the draw line or the return line is not fully primed; and may comprise, responsive to said determining, preventing operation of the pump.

According to a general aspect, a method for delivering gas-enriched blood within a vasculature of a patient is disclosed for priming of a blood circuit of a delivery system. The method comprises: performing, by a controller, a bidirectional priming of the blood circuit while a catheter is connected to the blood circuit, the controller configured for alternating a direction of blood flow through the blood circuit and alternating closure of first and second flow control mechanisms to alternatively block blood flow in a draw line a return line and prevent room air and/or air bubbles from flowing to the catheter during priming.

According to a general aspect, a delivery system for delivering gas-enriched blood within a vasculature of a patient is disclosed for priming of a blood circuit of the delivery system. The delivery system comprises: a blood circuit, comprising: a pump configured to circulate blood in the blood circuit in a forward direction or a reverse direction; a mixing chamber configured to mix blood of the patient with a gas-enriched liquid to form a gas-enriched blood; a draw line coupled to the mixing chamber and configured to connect a catheter to the mixing chamber and to interface with a draw clamp; a return line coupled to the mixing chamber and configured to connect the catheter to the mixing chamber and to interface with a return clamp; and a controller configured to perform operations comprising: activating the pump in the forward direction to circulate blood in the blood circuit to move blood up the draw line from a catheter in the vasculature of the patient toward the blood mixing chamber, the return clamp being closed on the return line to increase a pressure at the blood mixing chamber; venting air from the blood circuit to lower the pressure at the blood mixing chamber; activating the pump in a reverse direction to decrease a pressure in the return line of the blood circuit; opening the return clamp to enable blood to move up the return line to the blood mixing chamber and prime the return line; activating the pump in the reverse direction to move blood up the return line; and venting the air from the return line to prime the return line.

In some implementations, the system further comprises a control. The control may be configured to initiate operation of the controller to control operation of one or more of the pump, the return clamp, and the draw clamp to cause blood to flow in the reverse direction through the blood circuit through the mixing chamber to prime the return line and, optionally, to operate the pump in the forward direction to cause the blood to flow in the forward direction through the blood circuit to prime the draw line.

In some implementations, the control is a button. The operation of the controller may be initiated in response to a single press and/or release of the button. The button may be a physical button or a virtual button.

In some implementations, upon insertion of a cartridge comprising the mixing chamber into a cartridge housing of a console housing supporting the blood circuit, the draw line from the cartridge automatically self-aligns with the draw clamp positioned on the console, and, optionally, the return line automatically aligns with the return clamp positioned on the console.

In a general aspect, a blood delivery system includes a blood circuit comprising a pump configured to circulate blood in the blood circuit. The system includes a draw line coupled to the mixing chamber and configured to connect the catheter to the mixing chamber and to interface with a first flow control mechanism and a return line coupled to the mixing chamber and configured to connect the catheter to the mixing chamber and to interface with a second flow control mechanism. The system includes a controller configured to control operation of the pump and operation of the first and second flow control mechanisms to perform bidirectional priming of the blood circuit. The controller may be configured for alternating a direction of blood flow through the blood circuit and alternating closure of the first and second flow control mechanisms to block blood flow in the draw line and return line. As such, the controller may prevent room air and/or air bubbles from flowing to the catheter during priming.

According to a further aspect, which may generally relate to an earlier disclosed aspect, there is provided a gas-enriched blood delivery system for connecting to an intravenous catheter, the delivery system comprising: a blood circuit, comprising: a pump configured to circulate blood in the blood circuit; a mixing chamber configured to mix blood of the patient with a gas-enriched liquid to form a gas-enriched blood; a draw line coupled to the mixing chamber and configured to connect the catheter to the mixing chamber and to interface with a first flow control mechanism; a return line coupled to the mixing chamber and configured to connect the catheter to the mixing chamber and to interface with a second flow control mechanism; and a controller configured to control operation of the pump and operation of the first and second flow control mechanisms to perform bidirectional priming of the blood circuit. The controller may be configured for alternating a direction of blood flow through the blood circuit and alternating closure of the first and second flow control mechanisms to block blood flow in the draw line and return line. As such, the controller may prevent room air and/or air bubbles from flowing to the catheter during priming.

The system may comprise a removable cartridge.

The system may comprise a first vent and a second vent, wherein each vent is configured to vent room air and/or air bubbles from the blood circuit. The system may comprise the first flow control mechanism, which may be configured to control blood flow in the draw line to the catheter. The system may comprise the second flow control mechanism, which may be configured to control blood flow in the return line to the catheter. The controller may be configured to provide the bidirectional priming of the blood circuit by controlling operation of the pump, the first flow control mechanism, and the second flow control mechanism to perform operations comprising: causing the blood to flow in a first direction through the blood circuit wherein room air and/or air bubbles that are present in the blood circuit are removed from the blood circuit through the first vent to prime the return line; and causing the blood to flow in a second, opposite direction through the blood circuit wherein room air and/or air bubbles that are present in the blood circuit are removed from the blood circuit through the second vent to prime the draw line. As such, the bidirectional priming of the blood circuit may allow for priming of the blood circuit while the draw and return lines are connected to the catheter.

The first flow control mechanism may be a draw clamp. The second flow control mechanism may be a return clamp. The controller may be configured to perform operations comprising one or more of: activating the pump in the forward direction to circulate blood in the blood circuit to move blood along the draw line toward the blood mixing chamber while the return clamp is closed on the return line to increase a pressure at the blood mixing chamber; venting air from the blood circuit to lower the pressure at the blood mixing chamber; activating the pump in a reverse direction to decrease a pressure in the return line of the blood circuit; opening the return clamp to enable blood to move along the return line to the blood mixing chamber and prime the return line; activating the pump in the reverse direction to move blood along the return line; and venting the air from the return line to prime the return line.

In a general aspect, a non-transitory computer readable medium may be provided that comprises a computer program configured to cause one or more processors to perform the method of any preceding aspect.

According to a further aspect, which may generally relate to an earlier disclosed aspect, there is provided a cartridge for interfacing with a console, the console having a blood circuit configured for delivering gas-enriched blood within a vasculature of a patient via an intravenous catheter, the cartridge comprising one or more of: a gas-enrichment chamber configured to receive liquid and a source of gas for enriching the liquid to provide a gas-enriched liquid; a mixing chamber configured to mix the gas-enriched liquid with blood from a patient to form a gas-enriched blood, wherein the mixing chamber is configured to be connected to the blood circuit; and a bubble trap configured to be connected to the blood circuit and comprising a vent for removing room air and/or air bubbles from the blood circuit during a bi-directional priming process.

According to a further aspect, which may generally relate to an earlier disclosed aspect, there is provided a method for priming a blood circuit of a gas-enriched blood delivery system, the method comprising performing, by a controller, a bidirectional priming of the blood circuit by alternating a direction of blood flow through the blood circuit and alternating closure of first and second flow control mechanisms to alternatively block blood flow in a draw line a return line and prevent room air and/or air bubbles from flowing to the catheter during priming.

According to a further aspect, there is provided a method for bidirectional priming of a blood circuit comprising a mixing chamber configured to mix blood of the patient with a gas-enriched liquid to form the gas-enriched blood, a first vent, a second vent, a draw line, a return line, wherein the draw line and return line are configured to be connected to the catheter, the method comprising: causing blood to flow in a first direction through the blood circuit through the mixing chamber such that room air and/or air bubbles that are present in the blood circuit are removed from the blood circuit through the first vent to prime the return line; and causing the blood to flow in a second, opposite direction through the blood circuit through the mixing chamber such that room air and/or air bubbles that are present in the blood circuit are removed from the blood circuit through the second vent to prime the draw line.

According to a further aspect, there is provided a method for bidirectional priming of a blood circuit comprising a mixing chamber configured to mix blood of the patient with a gas-enriched liquid to form the gas-enriched blood, a draw line, a return line, and a catheter, wherein the draw line and return line are configured to be connected to the catheter, the method comprising: controlling a first flow control mechanism to close to prevent blood flow through the draw line; while the first flow mechanism is closed, causing a pump to circulate blood in a first direction through a bubble trap configured to remove room air and/or air bubbles from the blood circuit, the first direction being in a direction in the blood circuit toward the first flow control mechanism from the pump; controlling a second flow control mechanism to close to prevent blood flow in a return line; controlling the first flow control mechanism to open after the second flow control mechanism is closed; and while the second flow control mechanism is closed and the first flow control mechanism is open, causing the pump to circulate the blood in a second direction through the mixing chamber configured to remove room air and/or air bubbles from the blood circuit, the second direction being opposite the first direction, the second direction being in a direction in the blood circuit toward the second flow control mechanism from the pump.

According to a further aspect, there is provided a method for priming a blood circuit of a gas-enriched blood delivery system, the method comprising: activating a pump in a forward direction to circulate blood in the blood circuit to move blood along a draw line toward a blood mixing chamber while a clamp is closed on a return line to increase a pressure at a blood mixing chamber; venting air from the blood circuit to lower the pressure at the blood mixing chamber; activating the pump in a reverse direction to decrease a pressure in a return line of the blood circuit; opening a return clamp to enable blood to move along the return line to the blood mixing chamber and prime the return line; activating the pump in the reverse direction to move blood up the return line; and venting the air from the return line to prime the return line.

In a general aspect, a system may be provided that is configured to perform the method of any preceding aspect.

In some implementations, the system may comprise a controller that is configured to perform the method of any preceding aspect.

In general, an implementation or feature described with respect to one aspect may be implemented in combination with another aspect.

In general, the methods of priming the system may be performed ex vivo.

The details of one or more embodiments are set forth in the accompanying drawings and the description below. Other features and advantages will be apparent from the description and drawings, and from the claims.

### BRIEF DESCRIPTION OF THE DRAWINGS

FIG. 1 is a diagram of an example system that is configured for automatic priming to prepare the system for delivering gas-enriched blood within the vasculature of a patient.

FIG. 2A is a diagram of a portion of the system of FIG. 1 including a cartridge.

FIG. 2B is a diagram of a portion of the system of FIG. 1 including a cartridge.

FIG. 3 is a diagram of a piston device of the cartridge of FIGS. 2A-2B.

FIG. 4 is a diagram of an oxygenator of the cartridge of FIGS. 2A-2B.

FIG. 5 is a diagram of a blood mixing chamber of the cartridge of FIGS. 2A-2B.

FIG. 6 is a diagram of a bubble trap of the cartridge of FIGS. 2A-2B.

FIG. 7 is a perspective view of the system of FIG. 1.

FIG. 8 shows a flow diagram including an example process for automatically priming the system of FIG. 1 and FIG. 2A for delivering gas-enriched blood within the vasculature of a patient.

FIG. 9 shows a flow diagram including an example process for automatically priming the system of FIG. 2B for delivering gas-enriched blood within the vasculature of a patient.

FIG. 10 shows a flow diagram including an example process for automatically priming the system of FIG. 1 and FIG. 2B for delivering gas-enriched blood within the vasculature of a patient.

FIG. 11 shows a flow diagram including an example process for automatically priming the system of FIG. 1 for delivering gas-enriched blood within the vasculature of a patient.

FIG. 12 shows a flow diagram including an example process for automatically priming the system of FIGS. 1 and 2A for delivering gas-enriched blood within the vasculature of a patient.

FIG. 13 is a diagram of an example computing system.

### DETAILED DESCRIPTION

Described herein are various systems, methods, and catheters for delivering gas-enriched blood within the vasculature of a patient. The delivery system includes a blood circuit that adds gas-enriched liquid to blood in the blood circuit. The delivery system may include one or more catheters for delivering the gas-enriched blood, e.g., supersaturated oxygen (SSO₂) enriched blood, to a patient's vasculature and tissue. The delivery system is configured to automatically prime the blood circuit prior to delivery of the gas-enriched blood to the patient. The priming process may remove room air and/or air bubbles are from the blood circuit automatically (e.g., without human intervention after a user initiates priming, such as by actuating a priming actuator). The automated priming process includes a bidirectional priming process that removes room air and/or air bubbles are from both the return line and the draw line of the blood circuit, while both the return and draw lines are connected to the catheter.

As described previously, the benefits of the delivery system include the priming process being automatic and not requiring a user to interact with the delivery system after priming has commenced via a one-touch activation of the priming process. This is because the bidirectional priming process of the delivery system enables the blood circuit to be primed after the delivery system is connected to a catheter and after the catheter is placed inside the vasculature of the patient. Typically, gas enrichment systems require a wet connection being made between the return line and the catheter, where the return line is primed and filled with blood prior to connecting the return line to the catheter in the patient. This process can require skill, is time consuming, takes the user's attention away from the patient as the user must wait for blood to drip from the return line before connecting, and can waste blood and require cleanup in the patient's room. This process requires the user's full attention and prevents the user from attending to other tasks or caring for the patient in other ways. Such systems also require the priming button to be pressed and held throughout the duration of priming, requiring a first operator to hold the priming button and a second operator to perform the wet connection.

To overcome these disadvantages, the delivery systems described herein provides automatic bidirectional priming of the blood circuit via one-touch activation, by alternating pump direction and controlling blood flow through the draw and return lines using flow control mechanisms, such as clamps, valves, etc. Using a flow control mechanism to clamp or close the draw and return lines as described in this specification prevents air bubbles from escaping through the draw or return lines to the catheter in the patient, and allows priming to occur while both the return and draw lines are connected to the catheter. Because the draw and return lines are connected to the catheter during priming, no wet connection is required, and the problems associated with such a connection are avoided.

To provide automated bidirectional priming, the draw line is closed, and blood is pumped into the blood circuit through the return line. Blood is pumped through the return line, toward the draw flow control mechanism, and the return line is primed, as room air and/or air bubbles are vented from the circuit, while the flow control mechanism prevents room air and/or air bubbles from entering the patient's vasculature. Once the return line is primed, the return line is closed, the pump is reversed, and the draw line is opened. The pump reverses direction of blood flow in the blood circuit and blood is pumped through the draw line, toward the return flow control mechanism. The draw line is primed as room air and/or air bubbles are vented from the circuit and the return flow control mechanism prevents room air and/or air bubbles from entering the patient's vasculature. Once each of the return line and draw line are primed, and room air and/or air bubbles have been removed from the blood circuit, the system is ready to deliver gas-enriched blood to the patient, e.g., to perform supersaturated oxygen (SSO₂) therapy to treat ischemic tissue in a patient, e.g., in a patient who has suffered from a myocardial infarction. In certain embodiments, the above process may be reversed, where the draw line is primed prior to the return line.

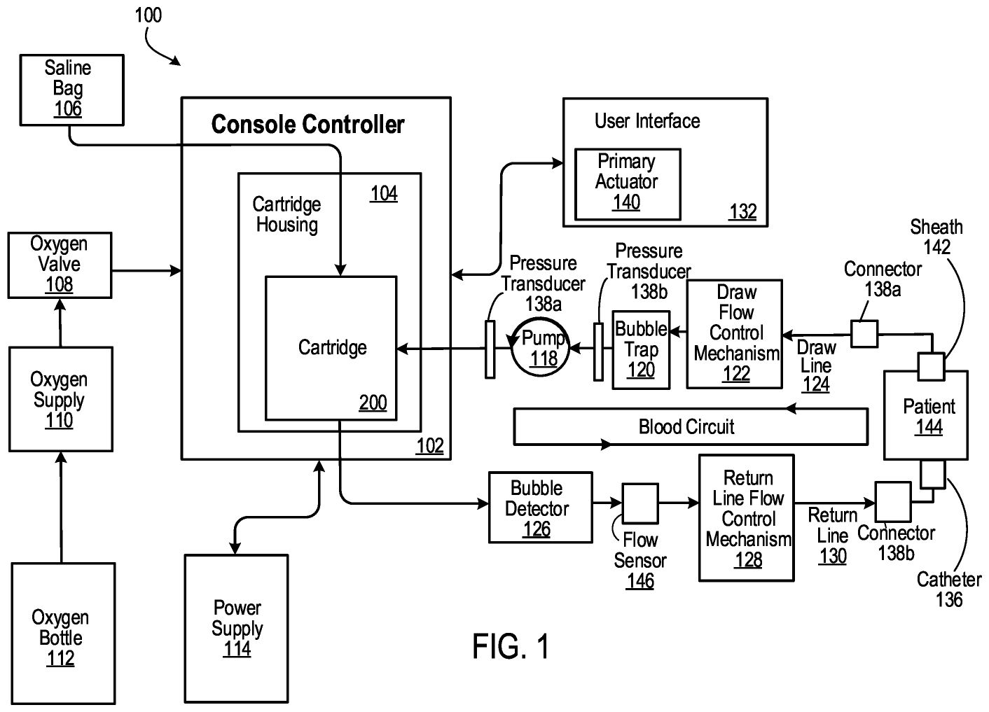

FIG. 1 shows an example delivery system 100 configured for automatically priming a blood circuit of the delivery system. The delivery system 100, once primed, can enable enrichment of a bodily fluid (e.g., blood) with a dissolved gas or gas-enriched liquid. As an example, the delivery system 100 creates a gas-enriched blood by enriching a patient's blood with a gas-enriched liquid, e.g., oxygen enriched liquid, in an extracorporeal gas-enrichment and control system including a controller 102 and a cartridge 200. Gas-enriched blood, e.g., oxygen enriched blood or supersaturated oxygen (SSO₂) enriched blood, is delivered to a patient 144, thereby increasing oxygen in the blood of the patient and diffusion of oxygen into tissue to treat ischemic (oxygen-deprived) tissue, e.g., in patients who have suffered a myocardial infarction.

The delivery system 100 is configured to prime a blood circuit of the delivery system prior to operation of the delivery system 100 to deliver the gas-enriched blood to the patient 144. More specifically, the delivery system 100 primes each of the draw line 124 and the return line 130 with blood prior to commencing gas enrichment of the blood and delivery of gas-enriched blood to the patient 144. Priming the blood circuit includes removing air pockets or air bubbles from each element of the blood circuit so that the entire or substantially the entire volume for each of these elements of the blood circuit is filled with blood.

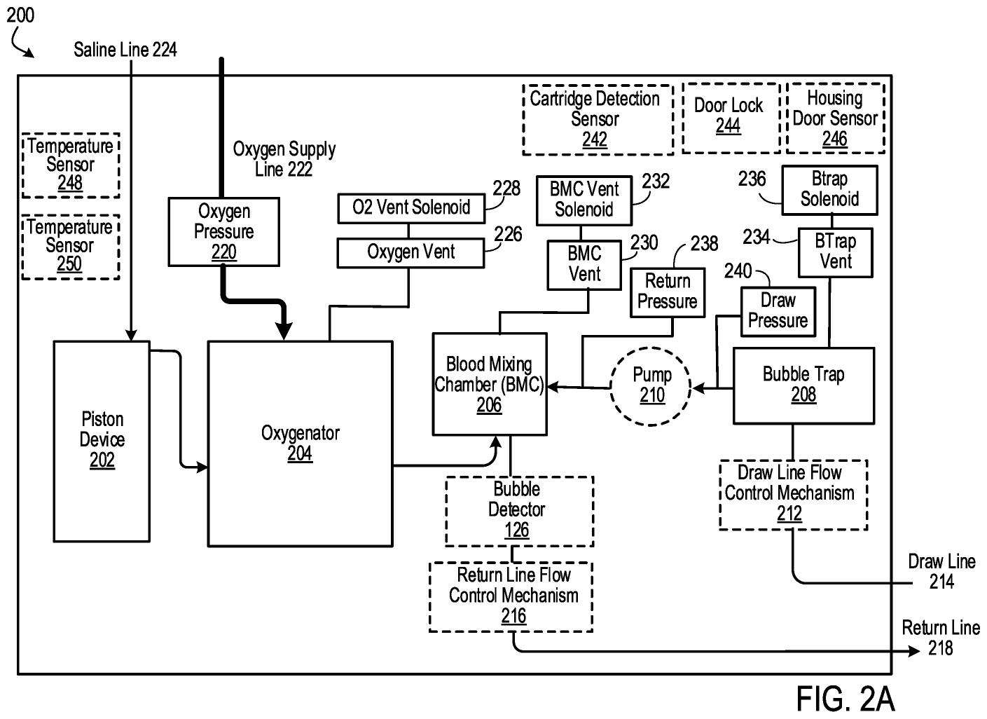

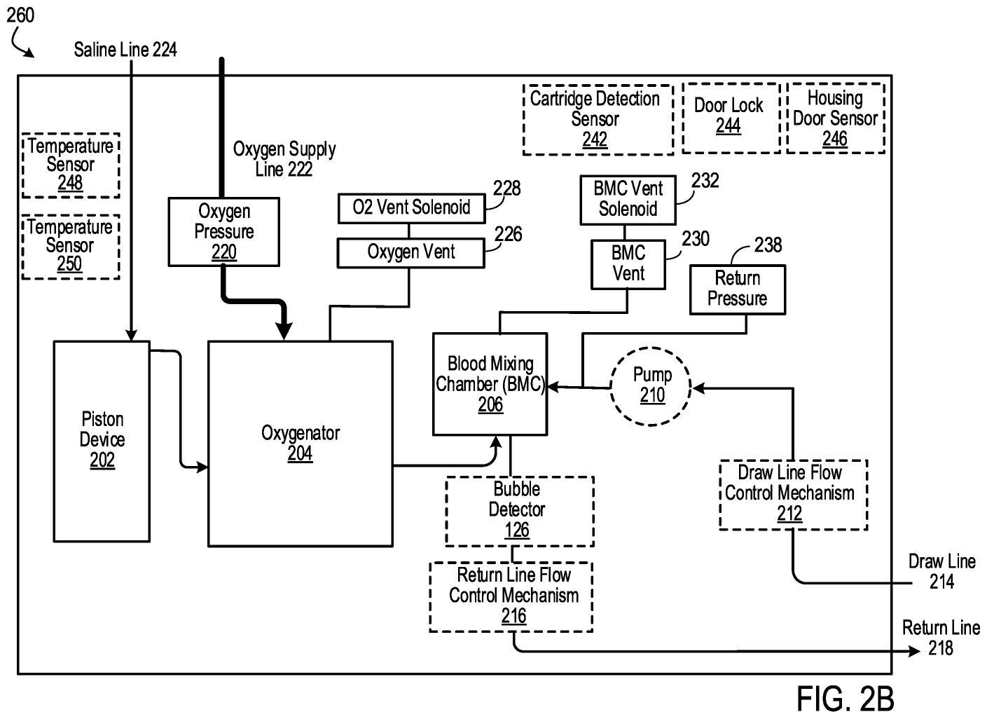

The blood circuit includes the blood mixing chamber of the cartridge that receives blood from the patient 144 and where enrichment of the blood with gas-enriched liquid occurs. The blood circuit may also include an air trap or bubble trap chamber. The blood circuit also includes the tubing between and among these chambers. The blood circuit of the delivery system 100 is connected to an intravenous catheter 136 which is insertable into the vasculature of a patient 144 to complete the blood circuit. Blood is removed from the patient 144, drawn into the cartridge of the delivery system 100, mixed with gas-enriched liquid, e.g., oxygen-enriched saline, and returned to the patient. The chambers of the blood circuit include one or more chambers of the cartridge 200, the bubble trap 120, and/or the bubble detector 126. The chambers of the cartridge are shown in FIG. 2 and described in greater detail with respect to FIGS. 2-6.

In certain embodiments, the delivery system 100 may include a console controller 102 cartridge housing 104, a user interface 132, a pump 118, a power supply 114, and an oxygen valve 108 and associated oxygen supply connector 110. The delivery system is configured to connect to several consumable items that are used as a part of the delivery system 100, including an oxygen bottle 112, fluid source 106 (or saline bag 106), a cartridge 200 and the catheter 136. Each of these elements is subsequently described in greater detail.

The delivery system 100 further includes a draw line 124 for drawing blood from a catheter 136 through connector 138a. The draw line 124 includes a bubble trap chamber 120 and is configured to interface with a pump 118 and a first flow control mechanism, e.g., a draw line flow control mechanism 122 of the delivery system 100. Pressure transducers 138a-b are may be located on either side of the pump 118 to measure pressure of blood flowing through the blood circuit, such as through the draw line 124, through the return line 130, or through each of the draw line and the return line.

The delivery system may include a flow sensor 146, for example on or near the blood circuit (e.g., on the return line 130) to measure the flow rate of the blood circulation in the blood circuit. For example, the flow sensor 146 can measure a number of milliliters per minute (mL/min) of gas-enriched blood delivered to the patient 144. In some implementations, the flow sensor 146 is positioned near the pump 118. In some implementations, the flow sensor is positioned near the return line 130.

The delivery system 100 includes a return line 130 for returning gas-enriched blood to the catheter 136 in the patient 144. The return line 130 is connected through a bubble detector 126, and connected to the catheter via a connector 138b. The return line is configured to interface with a second flow control mechanism, e.g., a return line flow control mechanism 128. As stated previously, a draw valve can be used to perform the functions of the draw flow control mechanism 122, and a return valve can be used to perform the functions of the return flow control mechanism 128. In some implementations, another mechanism for controlling or regulating flow of the blood in the blood circuit (e.g., to prevent blood flow and/or flow of room air or air bubbles as described) can be used to perform the functions of the draw flow control mechanism 122 or return flow control mechanism 128.

The catheter 136 is connectable to the delivery system 100. For example, the catheter 136 is a single-use consumable device that is used once before being discarded. The catheter includes a lumen for delivering gas-enriched blood to the patient 144. In the blood circuit, the draw line 124 may be connected (e.g., by connector 138a) to a sheath inserted into the patient 144 for drawing blood from the patient 144. The return line 130 may be connected (e.g., by connector 138b) to the catheter 136 to return blood to the patient 144. In some implementations, the catheter, which includes a lumen for delivery of the gas-enriched blood to the patient may be inserted through the sheath 142 positioned in the patient's vasculature. In this example, the return line 130 is connected to the catheter 136. The sheath includes a lumen for drawing blood from the patient 144, and the draw line 124 is connected to the sheath. Alternatively, the catheter may include a second lumen for drawing blood from the patient 144 so that the sheath 142 is not used, and so the catheter is configured for both returning and drawing blood from the patient 144. In this example, the draw line 124 and the return line 130 are connected to the catheter 136. The delivery system 100 is configured for use with different types of catheters. In another example, the sheath 142 includes a first lumen for connecting to the draw line 124 for drawing blood from the patient 144 and a second lumen for connecting to the return line 130 for returning blood to the patient.

To deliver gas-enriched blood to a patient 144, the delivery system 100 operates as follows. The delivery system 100 console 102 is connected to each other component of the delivery system. For example, the cartridge 200 is inserted into the cartridge housing 104 of the console 102. Tubing (e.g., the draw and return lines) extending from the cartridge and connecting the cartridge 200 to the catheter 136 is interfaced with the draw flow control mechanism 122, return flow control mechanism 128, and pump 118 of the console. The cartridge and draw and return lines 124, 130 may be configured such that upon insertion of the cartridge into the cartridge housing, the tubing automatically self-aligns with the draw flow control mechanism 122, return flow control mechanism 128, and pump. For example, the cartridge may have return and draw lines, which have a predefined orientation and shape that match with a corresponding shape or design in the cartridge housing and/or on the console. The predefined orientation and shape is such that upon insertion of the cartridge into the cartridge housing, the draw line and return line automatically align with and interface with the draw and return flow control mechanisms 122, 128, and the pump 118. The power supply 114 is connected to an external power source for providing power to the console 102. The oxygen supply 110 receptacle is provided an oxygen bottle 112 for providing the source of oxygen to the cartridge 200. The user interface 132 can indicate whether any of these consumables are missing from the delivery system 100 when priming is to begin.

Once each of the components of the delivery system are connected, including the cartridge 200, pump 118, bubble trap 120, bubble detector 126, draw flow control mechanism 122, return flow control mechanism 128, and catheter 136, the delivery system 100 is ready for use. The blood circuit is shown with arrows representing the direction of blood flow during operation of the delivery system 100, where blood is pulled from the catheter 136 through the draw line, through the cartridge and returned to the catheter via the return line.

In certain implementations, the delivery system 100 may enrich a bodily fluid with a dissolved gas or gas-enriched liquid, and deliver the gas-enriched bodily fluid, e.g., blood, to the vasculature and tissue of a patient 144 to treat ischemic tissue in the patient. As an example, the system 100 can be used to create a gas-enriched blood by enriching a patient's blood with a gas-enriched liquid, e.g., oxygen-enriched liquid, to form gas-enriched blood, e.g., oxygen enriched blood. The system 100 is configured to deliver the gas-enriched blood to a patient 144, e.g., in the case of oxygen, delivering oxygen enriched blood to a patient, thereby increasing oxygen in the blood of the patient and diffusion of oxygen into tissue. In certain implementations, gas-enriched liquid, e.g., oxygen-enriched liquid or solution, e.g., supersaturated oxygen liquid or solution, may include liquid, e.g., saline, having a dissolved O₂ concentration of 0.1 ml O₂/ml liquid (STP) or greater or 0.1-6 ml O₂/ml liquid (STP) or 0.2-3 ml O₂/ml liquid (STP) (e.g., without clinically significant gas emboli). When such supersaturated oxygen liquid or solution is mixed with blood, the resulting blood may be referred to as supersaturated oxygen enriched blood. In certain implementations, the system 100 may deliver an infusion of supersaturated oxygen enriched blood having an elevated pO₂ in a target range of 400 mmHg or greater or 600-1500 mmHg or 760-1200 mmHg or around 1000 mmHg.

In one example, supersaturated oxygen enriched blood may have a pO₂ of 760-1500 mmHg when a source blood delivered to the system for mixing with a supersaturated oxygen liquid or solution has a minimum pO₂ of 80 mmHg, the blood flow rate is 50-150 ml/min, the SSO₂ saline flow rate is 2-5 ml/min and the dissolved O₂ concentration in saline is 0.2-3 ml O₂/ml saline (STP).

In another example, where the source blood is below 80 mmHg, the treatment objective may be to boost the blood pO₂ to above 80 mmHg, so the system 100 may deliver an infusion of supersaturated oxygen enriched blood having a pO₂ level of 80 mmHg or greater or 80-760 mmHg.

Prior to operation the system 100 to deliver gas-enriched blood to a patient 144, e.g., supersaturated oxygen enriched blood, the system must be primed. During priming, the pump 118 is configured to reverse direction for bidirectional priming. The catheter 136 is inserted into the patient 144 at the desired location. Once the patient 144 and catheter 136 are set, the user initiates the automated priming process.

To initiate priming, a user can actuate a priming actuator 140. The user input can include a software control on the user interface 132 or display of the user interface. The priming actuator 140 can include a hardware button or switch. The actuator 140 can include a wireless signal, such as Bluetooth, Zigbee, WiFi, radio, or any such wireless transmission. The actuator 140 may include a mechanism that enables the user to initiate an automated “one-touch” priming process in which a single command is sent to initiate the priming process.

Once the priming process is initiated, the delivery system 100 is configured to perform the entire priming process automatically. The result of the priming process is that each element of the blood circuit is filled or substantially filled to threshold level with blood such that there is no room air and/or air bubbles in the blood circuit, which could travel to the patient 144. For example, the draw line 124 and return line 130 are filled with blood. For example, the bubble trap 120 and pump 118, and the tubing connecting them to each other and other elements of the blood circuit, are filled with blood. The blood mixing chamber of the cartridge 200 is filled with blood, e.g., to a threshold level.

Room air and/or air bubbles from each of the elements of the blood circuit is vented from the respective elements, as subsequently described. The bubble detector 126 is configured to detect any bubbles present in the blood circuit during operation of the delivery system 100 and can send a signal resulting in the closing of the return flow control mechanism 128 if room air and/or air bubbles are detected in the blood circuit. This prevents air bubbles from reaching the patient 144 at the catheter 136. The bubble detector 126 can include an ultrasonic sensor, infrared (IR) sensor (e.g., a photogate), or other such mechanism for detecting air or bubbles in line. For example, the bubble detector 126 can include an IR sensor that senses an IR beam sent through the fluid of the blood circuit. An air bubble in the fluid distorts the beam, which can be detected by an IR sensor.

To prime the blood circuit, the pump operates in a bidirectional manner, as controlled by the controller 102. In a first stage, the pump 118 operates in a first direction to draw blood through the return line 130 from the catheter 136. The draw flow control mechanism 128 is closed to prevent room air and/or air bubbles from entering the catheter 136 through the draw line. The pump 118 operates to draw blood into the blood circuit until the components of the blood circuit are filled with blood. At this point, the blood circuit is filled with blood up to the draw flow control mechanism 128, which is closed. The pump 118 may continue to operate until a desired pressure is reached in the pressure transducer 138b. The pressure measured by transducer 138b is indicative of whether there is any room air and/or air bubbles in the blood circuit on a return side of the pump 118. If the pressure exceeds a threshold, and/or one or more level sensors, not shown, show that the chamber of the bubble trap is filled with blood up to a level threshold, the blood circuit is known to have no remaining room air and/or air bubbles. Once all room air and/or air bubbles are removed, the pump 118 stops and the return flow control mechanism 122 is shut, preventing fluid flow back into the catheter 136 through the return line.

In a second stage, to prime the blood circuit, the pump 118 direction is reversed from the first stage. The pump is configured to fill the draw line 130 with blood in the second stage. The draw flow control mechanism 128 is opened once the return flow control mechanism 122 is closed. The pump 118 operates to pump blood toward the closed return flow control mechanism 122, or towards the return line 124. The draw line 130 and cartridge 200 are filled with blood from the catheter 136 drawn through the draw line 130. The pump 118 operates until the pressure transducer 138a reaches a threshold pressure and/or one or more level sensors, not shown, show that the blood mixing chamber is filled with blood up to a level threshold. The pressure measured by transducer 138a and/or the blood level measured by the one or more level sensors is indicative of whether there is any room air and/or air bubbles in the blood circuit on the draw side of the pump 118. Once all room air and/or air bubbles are removed, the return flow control mechanism 128 is open. In certain embodiments, the above process and stages may be reversed, where the draw line is primed and then the return line.

After these first two stages, the blood circuit is primed with blood from the patient 144 as the blood circuit is known to have no remaining room air and/or air bubbles. The delivery system 100 is ready to safely deliver gas-enriched blood to the patient 144 using the catheter 136. In some implementations, additional cycles of the first two stages are performed as a redundancy measure to ensure that no room air and/or air bubbles are present in the blood circuit.

The delivery system 100 may be configured to control the oxygen levels in the blood and/or tissues of the patient 144 by controlling the oxygen levels in the supersaturated oxygen liquid or solution, (e.g., targeting a dissolved O₂ concentration in saline of 0.2-3 ml O₂/ml saline (STP)) and/or the flow rate of the supersaturated oxygen enriched blood delivered to the patient 144, e.g., by controlling the speed of the pump to achieve a target blood flow rate of 50-150 ml/min. The system 100 may be configured to titrate oxygen into liquid e.g., saline, to be mixed with blood and adjust the oxygen level and/or blood flow rate, until the desired oxygen level is achieved (e.g., as measured by a blood oxygen sensor in the patient 144). In an example, the concentration of oxygen delivered, and/or blood flow rate may be modulated during treatment based on feedback from one or more sensors measuring various patient and/or system parameters.

One example of a sensor for measuring a partial pressure (pO₂) of oxygen or oxygen saturation SO₂ in the patient's blood is a pulse oximeter. A pulse oximeter may be used for estimating arterial pO₂ or SO₂. Pulse oximetry estimates the percentage of oxygen bound to hemoglobin in the blood. A pulse oximeter uses light-emitting diodes and a light-sensitive sensor to measure the absorption of red and infrared light. In another example, a sensor for measuring partial pressure of oxygen comprises an electrode such as a Clark electrode for measuring pO₂. A Clark electrode is an electrode that measures ambient oxygen concentration in a liquid using a catalytic platinum surface according to the net reaction O₂+4e⁻+4 H+→2H₂O. The various sensors may be coupled to a controller of the system via a cable or other wired connection or via a wireless connection.

The processor of a controller 102 can receive the signals from these sensors, which signals correspond to the measured values of pO₂. The processor compares the measured pO₂ to a target range of blood pO₂, e.g., 760-1500 mmHg. The target range can be calculated based on a source input blood pO₂ of 80 mmHg, a blood flow rate of 50-150 ml/min, an SSO₂ saline flow rate of 2-5 ml/min and dissolved O₂ concentration in saline of 0.2-3 ml O₂/ml saline (STP). The controller can adjust any of the above parameters based on the measured pO₂ in blood to achieve an arterial blood pO₂ within the target range. The processor may generate an alert, e.g., through a user interface, audible alarm and/or visual alarm that indicates the level of pO₂. The measured pO₂ indicates the effectiveness of the supersaturated oxygen therapy, letting the caregiver know if the pO₂ in blood is within the target range for optimizing the delivery of oxygen to the patient's ischemic tissue. In certain implementations, the processor may control the delivery of supersaturated oxygen therapy by modifying one or more of the above referenced saline or oxygen parameters based on the signals received from the sensors.

Another example of a sensor is an O₂ fluorescence probe. The fluorescence probe may be coupled to a controller of the system via a cable or other wired or wireless connection. A light source of the O₂ fluorescence probe is illuminated. A fiber optic cable can be used to provide light to the light source in certain implementations, where the fiber optic cable is connected to the controller of the system. The fluorescence of a sensor molecule of the O₂ fluorescence probe is measured. The sensor molecule can include fluorophore. A signal is received by the processor from the O₂ fluorescence probe based on the fluorescence measurement. Fluorescence is measured by measuring the lifetime or decay of the fluorescence intensity signal from the illuminated sensor molecule (e.g., fluorophore) on the fluorescence probe. The decay of this signal is caused by the quenching effect of oxygen molecules in the blood or in tissue on the fluorescence intensity signal of the sensor molecule. The processor can determine the oxygen concentration, SO₂ or pO₂ in blood or tissue based on the quenching effect of oxygen on the florescence intensity signal of the florescence probe. Changes in a time that is required for the signal to decay due to oxygen quenching are indicative of the local oxygen concentration, SO₂ or pO₂ in blood or tissue. The processor generates an alert, e.g., through a user interface, audible alarm and/or visual alarm, based on the determined oxygen concentration, SO₂ or pO₂ in blood or tissue. The alert may indicate the effectiveness of the supersaturated oxygen therapy. The determined oxygen concentration, SO₂ or pO₂ indicates the effectiveness of the supersaturated oxygen therapy, letting the caregiver know if the oxygen concentration, SO₂ or pO₂ in blood is within a predefined target range (e.g., the expected range for a healthy individual) for optimizing the delivery of oxygen to the patient 144. In certain implementations, the processor may control the delivery of supersaturated oxygen therapy by modifying one or more of the saline or oxygen parameters, e.g., saline flow rate or dissolved O₂ concentration in saline, based on the determined oxygen concentration, SO₂ or pO₂ values.

The user interface 132 is configured to display operational data and/or patient data on the user interface in a configuration that allows a user to determine a status for the SSO₂ liquid and gas-enriched blood delivery to the patient 144. The user interface 106 shows a current operational status of the delivery system 100.

These values can be stored as a time sequence of data entries or log entries in an operational log. The user interface may include a visual representation of the operational log, the visual representation including operational data specifying how the delivery system 100 is performing during priming and after priming is completed. For example, the delivery system 100 logs sensor readings during priming and generates an alert or report indicating a successful priming process has completed. In some implementations, the delivery system 100 logs data indicating a transition from a priming state to a therapy delivery state, which is an indication that the priming process was successful. In some implementations, the delivery system 100 can send logged data to remote, networked storage (e.g., in cloud storage) for access from one or more networked devices.

In some implementations, various data elements are logged during the automated priming process. For example, the duration of priming can be logged. Each time a checkpoint is reached, a time stamp associated with the checkpoint is saved. Checkpoints can include completion of the priming process, reversal of the pump, validation of room air and/or air bubbles being removed from one or more components (such as tubing, the air trap or the blood mixing chamber), or any other point of interest during the priming process. The values of sensors, such as the level sensors, pressure sensors and temperature sensors, can be stored at given instances in time. The operational values of devices on the blood circuit can be monitored, such as how fast the pump is operating, blood levels in the bubble trap, when the flow control mechanism (e.g., a draw clamp or a return clamp) are actuated, and so forth. These data provides information to determine whether an issue is occurring during priming. For example, if the return flow control mechanism and the draw flow control mechanism do not open and close in an expected order, a fault item or other error value can be included in the log. The delivery system 100 can determine if the time spent on a stage of priming exceeds an expected time by a threshold amount and/or identify an error indicating that a problem with the priming process may be occurring. Similarly, if a stage of the priming process is far shorter than expected (e.g., below a threshold percentage of an expected range of times), the system can flag the data as representing a potential error. The delivery system 100 can obtain data describing operation of valve and vents of the components of the blood circuit to determine whether room air and/or air bubbles are venting from the circuit as expected. In certain implementations the data that may be recorded and/or logged includes delays in any portion or stage of the priming sequence and/or data regarding functional, safety or operation checks of various sensors or valves in the system.

In some implementations, the delivery system 100 may include a processor, a memory, and associated circuitry coupled to the one or more sensors for detecting operational or patient data. The operational and patient data are collected and/or stored in the system for retrospective, current or other review. The delivery system 100 is configured to generate log entries for the operational data (e.g., priming data). The log entries may be displayed on the user interface 132. In certain implementations, the log entries can each be structured messages that include particular values associated with the operation of the delivery system 100, generated from data messages. In some implementations, a data message (also called a log message) represents an instant snapshot of the operational data. For example, a data message can include priming data or current pO₂ and SO₂ values at a given time (e.g., associated with a time stamp). In some implementations, a data message can include data representing priming, a treatment period or system mode of the gas-enriched liquid treatment for the patient 144 in a structured log entry. The data messages are stored in a digital format that enables streaming of the data messages to a remote system. The remote system is configured to quickly extract the values representing the patient data and the operational data of the delivery system 100 and display a representation of these data on a local or remote user interface. For example, data messages can be formatted for streaming to an operator or nurse's station from a hospital room. In some implementations, data messages can include warnings or alerts that prompt intervention from a user of the remote system. In some implementations, the data messages can be stored in a structured format that facilitates searching and retrieving of operational data for the patient 144 for operation of the delivery system 100 during priming and after priming is completed.

In some implementations, the log entries can each be structured messages that include particular values associated with the operation and/or priming of the delivery system 100, generated from data messages. For example, the data messages can indicate a current snapshot of the operation of the delivery system 100. In this case, the values of the data message include a list of operational values (and in some implementations, SO₂ and/or pO₂ data). The operational values can be parsed from the data messages (e.g., by a remote device) and used to populate a screen or display of a remote computing system. For example, the delivery system 100 can transmit a stream of data including the data messages to a remote system for remote monitoring of the operation of the delivery system 100. In some implementations, the processor is configured to stream digital output data having the patient data and the operational data to a remote server. In some implementations, operational and patient data may be transmitted or streamed in real time or near real time via a wired, RS-232 streaming output on the system console to a remote processor or computer, e.g., to an EMR data hub or hospital hub. In some implementations, operational and patient data may be transmitted or streamed in real time or near real time over a WiFi communications, Bluetooth, cellular, USB or other wireless connection or link.

The data messages can include summary data. For example, log entries can include data representing a summary of operational data for a time period (e.g., pre-priming data, priming data, and post priming data). Each log entry may form all or a portion of the operational log, which provides an overall summary of the operation of the delivery system 100. The operational log allows a medical service provider to quickly review the summary of the operation of the delivery system 100. The operational and patient data, e.g., data messages, log entries, operational log and/or other data, stored by the system processor or an accessary to the system or data module, coupled to the system console, may be stored on volatile or non-volatile memory. The log entries can be visually represented on the user interface 132.

Data messages may provide instant values of operational data of the delivery system 100 and the patient data. Log entries may represent data gathered over time and can be part of a system and/or patient profile. For example, the operational log and the log entries can be stored in electronic medical records (EMR).

In some implementations, the log entries of the operational log are transmitted to a remote device (such as a data hub in a hospital). The delivery system 100 sends the data including the log entries to the remote device in one or more different ways. The delivery system 100 sends the log entries data to a remote device in response to a trigger. For example, the delivery system 100 can send the log entries to the remote device once priming is completed. In some implementations, the delivery system 100 sends the operational log data once all treatment is completed. For example, when the cartridge 200 is removed or the pump 118 is powered off, the controller 102 can determine that treatment is completed and send the log entry data to the remote device.

In some implementations, the delivery system 100 sends the operational log data to the remote device upon detecting the presence of an air bubble during priming or upon detecting a fault, such as a bubble trap 120 fault, a catheter 136 fault, a patient SO₂ or pO₂ value failing a threshold, etc. The operational log data can be analyzed (e.g., by a user) to determine why the fault occurred and/or to determine whether operation of the delivery system 100 is adversely impacted by the fault. This enables the user to take corrective measures immediately (e.g., replacing a bubble trap 120, fixing a fluid leak, etc.) to ensure that priming or a treatment of the patient 144 is not compromised.

In some implementations, the delivery system 100 sends the operational log data without a trigger. For example, the delivery system 100 can send the log entry data to the remote device periodically (e.g., once per minute, once per hour, etc.).

In an aspect, the delivery system 100 links the log entries related to operation and/or priming together in a structured format. For example, a key value can be stored with each log entry. The entire log of the operation of the delivery system 100 can be retrieved by referencing the key value.

The delivery system 100 can generate one or more alerts to indicate a status of one or more components of the delivery system 100. The alerts can be generated based on the operational log data or data of the data messages. The alert can be generated for presentation on a user interface 132 of the delivery system 100. The processor may send the alert to one or more other computing devices, such as computing devices associated with a health care provider of the patient 144. In an aspect, a user interface is configured to communicate with the processor, wherein the data representing the alert indicating whether a fault has occurred, priming has initiated/completed, or any other relevant aspect of the operation of the delivery system 100 that satisfies a notification rule causes a notification to be displayed on a user interface. The user interface may be coupled to the console via a wire or wirelessly (e.g., the user interface may be a portable tablet or remote computing device)

The alert may indicate that there is a fault or error in operation of the delivery system 100. The alert provides an indicator for a health care provider to investigate the operation of the delivery system 100, such as to investigate whether any faults have occurred. The alerts may indicate that priming has completed, that there is a pressure over value in the blood circuit, that room air and/or air bubbles have been detected, etc.

In some implementations, the processor generates the alert to cause one or more devices to perform an action. For example, feedback can be presented to a healthcare provider, such as an audio cue, visual presentation, and so forth. The alert can cause a device to contact a healthcare provider (e.g., place a phone call or page to a physician, nurse, etc.). The alert can cause a device to display particular data about the priming process or performance of the system, or data about the patient 144, such as a presentation of the patient's SO₂ and/or pO₂ values over a given treatment period. The alert can cause a device to update a health record associated with the patient 144 or cause the device to retrieve a health record associated with the patient for further analysis. In certain implementations, the processor of the system may be configured to determine if the alert is a real time alert or recorded for retrospective review. If it is a real time, the processor determines whether to display the alert on the user interface, transmit the alert in an information chain, or send the alert data to a third-party monitor. An example route is to send the alert to a physician or nurse's cell phone.

The alert may open a cell phone-based application or open an Internet-based application. From either application the physician or nurse could see the alert plus other relevant data that may have been transmitted. The alert may include a hospital specific patient identifier, but otherwise be invisible as to the identity of the patient 144, unless the physician or the hospital has added the patient's name to either the application on their phone or to the Internet. The alert may include a non-patient specific identifier such as a bed number. Additionally, the physician would have the opportunity to take actions in response to receiving the alert. This might include triggering a phone call to the ICU desk or marking that the physician has seen the alert. Changing the duration or range of a monitored value would allow the user to set a duration so that a transient spike would not trigger the alert. In the case of adjusting the time and/or duration of the alert, such an adjustment may only affect the notification to that specific person.

A dual alert to a nurse or physician might have different alert ranges and actions. The described features may put the user, e.g., physician in complete control. For example, the first point of control may be at the bedside, where the alert ranges may be set. The second point of control may be at the receiving application or website where the user may adjust nominal settings, e.g., for “tones”. As such, two or more triggers may be established: the first is to “send” the alert from the machine into the network to the receiving device; and the second is the action that the receiving device takes upon receiving the alert. A scheduling feature may also be provided that allows for the transfer data from one physician going off shift to another coming on shift. A response tree may be provided that requires an acknowledgement that the alert has been seen or transferred from one physician to another. For example, a first doctor is given 5 minutes to acknowledge receipt of the alert, and if no acknowledgment is made, the alert is sent to another physician or nurse. In certain implementations, one or more of the various alerts or alert parameters described herein may be customized by the user. Multiple options for alert delivery, e.g., device display, nurse's station, EMR, cell phone, etc. may be set. An alert for thermoregulatory activity of a patient 144 may include other forms. For example, a color scale or audible alert may be output via the user interface to provide a value indicative of patient activity.

In some implementations, a medical service provider can query the delivery system 100 to obtain the operational data. The query can request particular data, such as what the battery status is, determine whether priming was successful, and so forth. When the priming is completed, the delivery system 100 reports a successful priming operation and that treatment is initiating.

In some implementations, a controller is configured to store digital output data representing the priming process in a data store. The controller is configured to detect that a trigger condition of the priming process is satisfied. For example, the trigger condition can include completion of all or a portion of the priming process. In some implementations, the controller, in response to detecting that the trigger condition is satisfied, transmits the digital output data to a remote device in real time or in near real time, e.g., during or after the priming of the delivery system.

In some implementations, the digital output data includes a predefined format that enables the digital output data to be streamed to a remote device. The delivery system can include a transmitter configured to transmit the digital output data to the remote device. In some implementations, the predefined format is configured to enable the remote device to parse the digital output data for displaying the priming data, the operational SO₂ or pO₂ data and/or the operational data upon receiving the digital output data. In some implementations, the process 800 includes streaming the digital output data over a WiFi communications, Bluetooth, cellular, or other wireless connection or link or USB. In some implementations, the process 800 includes transmitting the digital output data over a wired connection.

FIG. 2A is a diagram of an example of a portion of the system of FIG. 1 including the cartridge 200. In this example, the cartridge 200 includes a fluid supply chamber (piston device 202), a gas enrichment chamber (an oxygenator 204), and a blood mixing chamber 206. In some implementations, the cartridge 200 may also include a bubble trap 208, and at least a portion of the draw line 214 tubing and the return line 218 tubing. In FIG. 2A, the pump 210 is similar to pump 118, the draw line 214 is similar to draw line 124, the return line 218 is similar to return line 130, and the bubble trap 208 is similar to bubble trap 120. The cartridge 200 is consumable portion of the blood circuit that includes portions of the blood circuit that contact the patient's blood. The return draw flow control mechanism 216, pump 210, and draw flow control mechanism 212 are shown in dashed lines because these are a part of the console system and are reusable. Similarly, the return pressure sensor 238 and/or the draw pressure sensor 240 are reusable; however, in certain embodiments, the return pressure sensor 238 and/or the draw pressure sensor 240 may be part of the single use consumable cartridge and tubing.

The controller 102 initiates a self-test in which the system 100 tests the cartridge 260, flow control mechanisms 216 and 212 (e.g., a return line clamp and a draw line clamp), a valve or vent 232 in the blood mixing chamber 206 and the vent 240 of the bubble trap 120, the pump 210, and the pressure sensors 238 and 240. These sensors and devices are tested for correct operation and responsiveness to control signals from the controller 102. The system 100 determines automatically whether a sensor or hardware device has failed to ensure that no air is advanced to the patient. Once the priming sequence is completed, the controller 102 also determines whether any air is remaining in the draw line 214 or the return line 218, as previously described, to ensure that the priming process is successful and to ensure that no air is advanced to the patient.

The cartridge 200 is configured to interface with components of the console 102 of the delivery system 100 during operation, priming and treatment. A portion of the tubing of the cartridge 200, which can be called a pump tube, is configured to be placed in the pump 210 of the console. The draw line 214 tubing and the return line 218 tubing are oriented to be placed inside the draw flow control mechanism 212 and the return flow control mechanism 216, respectively. The flow control mechanisms 212, 216 are coupled to the console 102. When the cartridge 200 is installed, the flow control mechanisms 212, 216 align with the draw and return lines 214, 218 to enable the flow control mechanisms to restrict fluid flow (e.g., by clamping) in the draw and return lines 214, 218. The draw flow control mechanism 212 and the return flow control mechanism 216 are actuated by control signals of a controller of the console 102. Similarly, the pump 210 is coupled to the console 102. The pump 210 is activated by control signals of the controller of the console for pumping in either the draw line direction or the return line direction during the bidirectional priming process.

To install the cartridge 200, the cartridge is inserted into a housing 104 (of FIG. 1). The housing 104 includes a housing door sensor 246, a door lock 244, a cartridge detection sensor 242, and temperature sensors 248, 250. The housing door sensor 246 reports whether the cartridge housing 104 door is open or closed. If open, operation of the delivery system 100 is paused by the controller of the console 102, which receives signals from the door sensor 246. Similarly, the cartridge detection sensor 242 reports whether the cartridge 200 is present in the housing 104. If not present, the controller of the console 102, which receives signals from the detection sensor 242, causes a notification to be displayed to the user on interface 132. The door lock 244 can be actuated by a signal from the controller of the console 102 (e.g., an electromagnet). The door lock 244 retains the cartridge housing door closed during operation of the delivery system.

When the cartridge is inserted into or otherwise coupled to the console 102, the shape of the cartridge 200 and the console receptacle for the cartridge or cartridge housing is shaped to guide the cartridge into the correct orientation in the console housing 104. This aligns the return flow control mechanism 216 with the return line 218, the draw flow control mechanism 212 with the draw line 214, and the pump 210 with the pump tubing between the blood mixing chamber 206 and the bubble trap 208. The temperature sensors 248, 250 are configured for measuring saline temperature. Optionally, the system may include temperature sensors for measuring blood temperature in each of the draw line and the return line. As discussed supra, the cartridge and draw and return lines may be configured such that upon insertion of the cartridge into the cartridge housing, the tubing automatically self-aligns with the draw flow control mechanism 212, return flow control mechanism 216 and pump 210. For example, the cartridge may have return and draw lines, which have a predefined orientation and shape. The predefined orientation and shape is such that upon insertion of the cartridge into the cartridge housing, the draw line and return line automatically align with and interface with the draw and return flow control mechanisms 212, 216, and the pump 210. The cartridge housing may also be shaped to receive the cartridge in a single orientation, which aligns the draw and return lines 214, 218 with the draw and return flow control mechanisms 212, 216 and seats the pump tubing in the pump 210.

The saline line 224 is configured to connect to the fluid source 106 of FIG. 1, which can include an intravenous saline bag (IV saline bag). The draw line 214 may be connected to a catheter, and the return line may be connected to the catheter. The catheter is inserted into the patient prior to priming the delivery system 100. Alternatively, the catheter may be inserted through a sheath positioned in the patient's vasculature. The sheath may include a lumen for drawing blood from the patient, where the draw line is connected to the sheath.

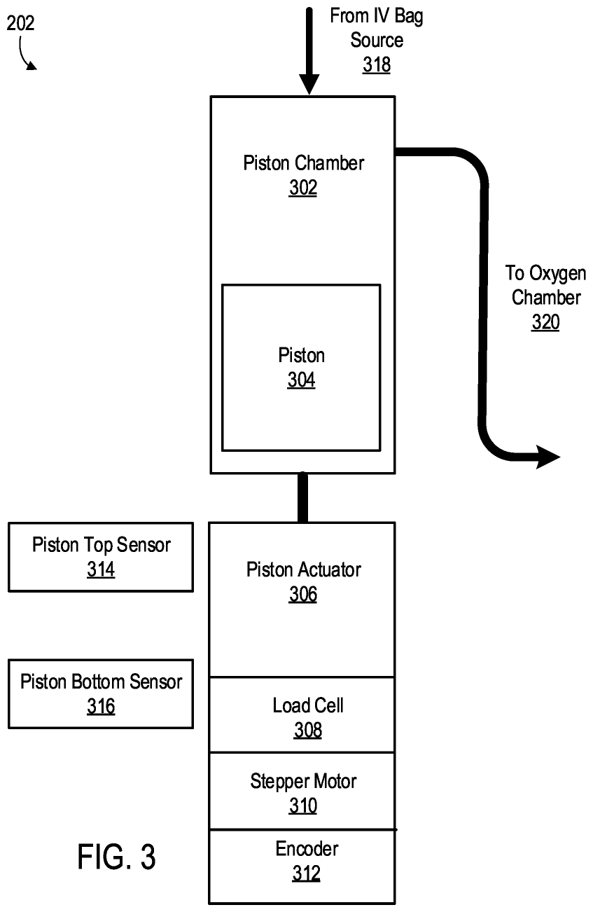

The piston device 202 includes a mechanical device for drawing saline from the fluid source. The piston device 202 is shown in greater detail in FIG. 3. As shown in FIG. 3, the fluid from the IV source is drawn through tubing 318 into a piston chamber 302. The piston 304 moves vertically in the chamber 302 based on signals from a piston actuator 306. A load cell 308 determines the force required to move the piston 304. A stepper motor 310 controls the motion of the actuator 306. An encoder 312 reports the piston position based on the stepper motor 310 rotor location. A piston top sensor 314 and piston bottom sensor 316 can detect when the piston moves to an edge of the chamber 302. The position of the piston determines how much fluid from the saline bag is sent to the oxygenator, e.g., through tubing 320.

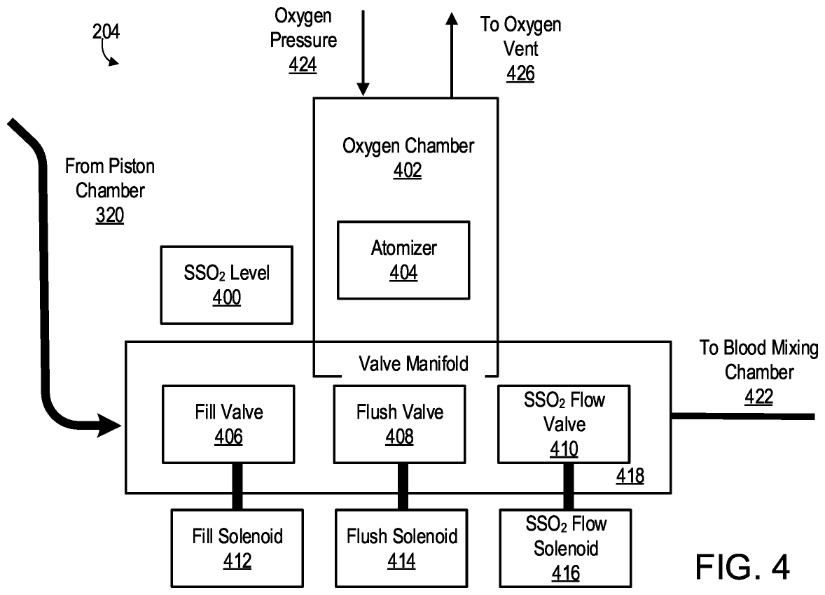

Returning to FIG. 2A, the piston device 202 is configured to draw saline into the oxygenator 204. The oxygenator 204 is described in additional detail with respect to FIG. 4. The oxygenator 204 is configured to add oxygen to the saline from the saline bag 106. An oxygen pressure line 220 adds oxygen to the oxygenator 204. The oxygenator 204 is coupled to an oxygen vent 226 and an oxygen vent solenoid 228 that controls operation of the vent 226. The oxygenator vent 226 is configured to vent excess air from the oxygenator if the oxygen pressure exceeds a threshold value.

Turning to FIG. 4, the oxygenator 204 is shown in greater detail. The oxygenator 204 includes an oxygen chamber 402, an atomizer 404, and a valve manifold 418. The valve manifold includes several valves such as a fill valve 406, a flush valve 408, and a supersaturated oxygen SSO₂ flow valve 410. Each of the fill valve 406, flush valve 408, and SSO₂ flow valve 410 are controlled by a respective solenoid 412, 414, and 416. The fill solenoid 412 opens/closes the fill valve 406. The flush solenoid 414 opens/closes the flush valve 406. The SSO₂ flow solenoid 416 opens/closes the flow valve 410. An SSO₂ level sensor 400 indicates a level of the gas-enriched liquid in the oxygenator.

The oxygen chamber 402 is connected to the oxygen pressure line 424 and the oxygen vent 426. The oxygenator releases excess oxygen through oxygen vent 426 and receives additional oxygen through oxygen pressure line 424. The oxygenator receives fluid from the piston chamber e.g., via tubing 320, into the valve manifold 418.

The atomizer 404 includes a central passageway in which a one-way valve is disposed. When the fluid pressure overcomes the force of the spring in the one-way valve and overcomes the pressure of the oxygen within the atomizer chamber, the fluid travels through the passageway and is expelled from a nozzle at the end of the atomizer.

The nozzle forms fluid droplets into which the oxygen within the atomization chamber diffuses as the droplets travel within the atomization chamber. This oxygen-enriched fluid is referred to a SSO₂ solution. The nozzle is preferably a simplex-type, swirled pressurized atomizer nozzle including a fluid orifice of about 0.004 inches diameter to 0.005 inches diameter. The droplets infused with the oxygen fall into a pool at the bottom of the atomizer chamber. Since the atomizer will not atomize properly if the level of the pool rises above the level of the nozzle, the level of the pool is controlled to ensure that the atomizer continues to function properly

Once the oxygen has been dissolved into the saline using the controlled pressure, the gas-enriched saline is sent to the blood mixing chamber 206 for mixing with blood in the blood circuit.

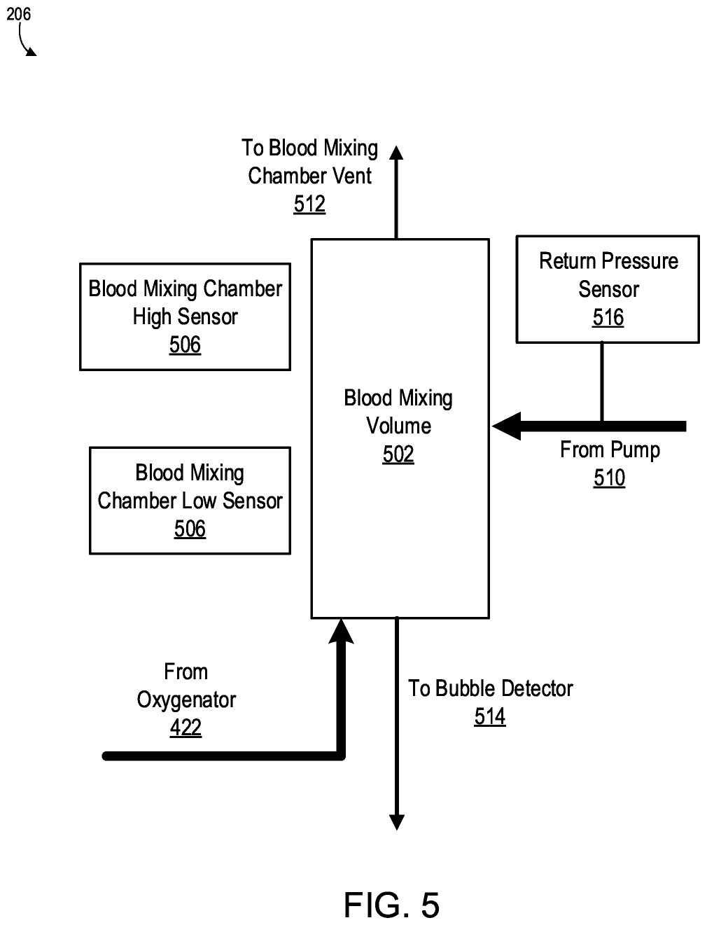

Returning to FIG. 2A, the blood mixing chamber 206 is connected to the oxygenator 204. The blood mixing chamber 206 is thus a part of the blood circuit. The blood mixing chamber 206 is positioned between the pump 210 tubing and the return line flow control mechanism 216 and bubble detector 126. A blood mixing chamber 230 is configured to vent any room air and/or air bubbles from the blood mixing chamber 230. A blood mixing chamber vent solenoid 232 controls operation of the vent 230. The blood mixing chamber is shown in greater detail in FIG. 5.

FIG. 5 shows the blood mixing chamber 206 in greater detail. The blood mixing chamber 206 includes a volume 502 configured to receive gas-enriched saline from the oxygenator 204. The blood mixing chamber 502 includes low sensor 504 and a high sensor 506. The low sensor is configured to detect when the blood mixing volume 502 is empty. The high sensor 506 detects when the blood mixing volume 502 is full.

The blood mixing volume 502 vents room air and/or air bubbles from the blood circuit through the vent 232 through the line 512. The blood mixing chamber receives gas-enriched saline from the oxygenator, e.g., through line 422. The blood mixing chamber receives blood from the pump 210 from the pump tube 510 during operation of the delivery system 100. The gas-enriched saline from the oxygenator 204 mixes with the blood from the draw line of the blood circuit. A return pressure sensor 516 measures pressure in the blood circuit on the return line side of the pump 210. The blood from the blood circuit passes through the blood mixing volume and mixes with the gas-enriched saline from the oxygenator 204. The return line draws blood out of the blood mixing volume 502 to the bubble detector 514.

Returning to FIG. 2A, the blood mixing chamber 206 oxygenator and piston chamber may be located in a single housing or separate from one another. The pump 210 is configured to interface with a pump tube. The pump tube connects the bubble trap 208 to the pump 210. The pump tube connects the blood mixing chamber 210 to the pump on of the opposite side of the pump 210 from the bubble trap 208. Blood in the blood circuit during operation of the delivery system 100 thus comes from the draw line 214 through the bubble trap, is pumped by the pump 210, goes through the blood mixing chamber 206, and then goes through or passes by the bubble detector 126 in the return line 218. During priming, the draw flow control mechanism 212 and return flow control mechanism 216 are alternately closed so that no blood or room air and/or air bubbles are pushed through the draw or return line and the pump can be run forward or in reverse. During delivery of gas-enriched blood to the patient, both the draw flow control mechanism 212 and the return flow control mechanism 216 are open.

When the pump 210 reverses direction to prime the return line 214, and the order of the blood through the blood circuit is reversed. Blood comes through the return line 218, through or by the bubble detector 126, through the blood mixing chamber 206, through the pump 210, and through the bubble trap 208 until all the room air and/or air bubbles have been removed. The draw flow control mechanism 212 is closed while the pump 210 operates in reverse so that no blood is pushed through the draw line 214 beyond the draw claim and to the patient.

The bubble trap 208 is configured to remove room air and/or air bubbles from the blood circuit. During priming, the pump 210 pumps blood through the bubble trap 208 in each of a first direction in the first phase to prime the return line 216 and a second direction in the second phase to prime the draw line 214, as previously described.

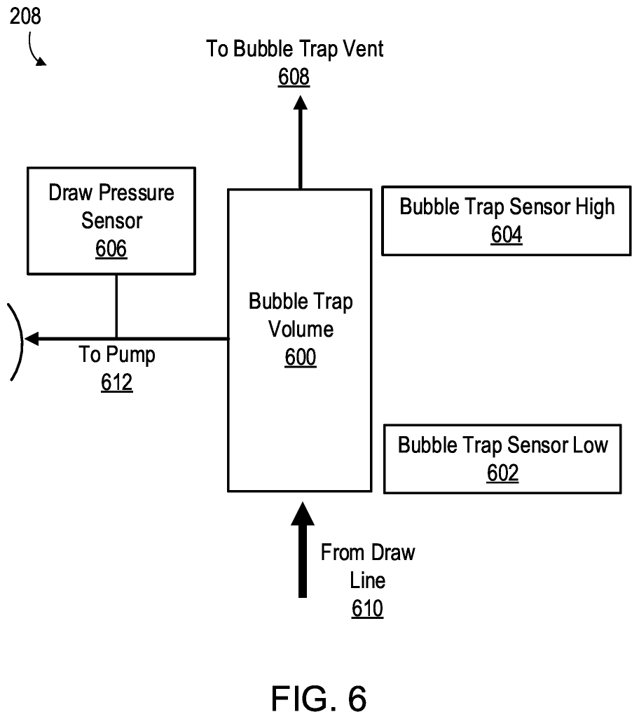

FIG. 6 shows the bubble trap 208 in detail. The bubble trap 208 has a bubble trap volume 600 configured to receive blood from the draw line 610. The bubble trap volume 600 vents room air and/or air bubbles from the volume to the bubble trap vent 608. Bubbles rise to the top of the volume 600 and are vented. The bubble trap volume 600 has a low sensor 602 to detect when the bubble trap volume 600 is empty. The bubble trap volume 600 has a high sensor 604 to detect when the bubble trap volume is full. When the volume 600 is full of blood, the bubble trap 208 is primed. Blood from the draw line 610 passes through the volume 600 and to the pump tube 612 to the pump 210. A draw pressure sensor 606 measures blood pressure on the draw line side of the pump 210.

Returning to FIG. 2A, the delivery system 100 is configured to prime each of the blood mixing chamber 206, the bubble trap 208, and the tubing of the cartridge 200 with blood using the automatic, bidirectional, two-phase priming process. In the first phase, the pump 210 is configured to pump 210 blood toward the closed draw flow control mechanism 216 in the blood circuit. The pump 210 pumps blood until a return pressure 238 exceeds a threshold. The relatively high pressure in the return side of the blood circuit forces room air and/or air bubbles out of the vent(s) of the bubble trap 208. The pump 210 thus causes room air and/or air bubbles in the blood circuit to be removed. Once the pressure measured by the sensor 238 reaches or exceeds a threshold and/or the blood level detected by the level sensor in the bubble trap is reached or exceeded, the controller 102 of the delivery system 100 closes the return flow control mechanism 216 and reverses the direction of the pump. The return line 216 is full of blood beyond the return flow control mechanism 216.

The controller 102 opens the draw flow control mechanism 212. The draw line 218 can still have room air and/or air bubbles in it and is now primed with blood from the catheter 136. The pump 210 pumps blood toward the closed return line flow control mechanism 216 in an opposite direction. Blood is drawn through the draw line 214 and through the blood mixing chamber 206. Once the pressure measured by the sensor 240 reaches or exceeds a threshold and/or the blood level detected by the level sensor in the blood mixing chamber is reached or exceeded, the draw line 218 has been primed. The room air and/or air bubbles are removed by the blood mixing chamber vent 230. While two pressure sensors 238, 240 are shown in FIG. 2A, and only one pressure sensor 238 is shown in FIG. 2B, the methods described in this specification are performable with either a single pressure sensor or two or more pressure sensors. For example, the system 260 of FIG. 2B can include two pressure sensors 238 and 240, and the system 200 of FIG. 2A can include a single pressure sensor 238.

Another important benefit provided by the bubble trap in the blood circuit may be realized during operation of the delivery system 100 when the system is delivering oxygen-enriched blood to a patient, after the system has been primed. During system operation, if there's a problem, e.g., an air bubble is detected in the blood circuit, the air bubble will be captured in the bubble trap. For example, if during blood sampling, air bubbles inadvertently enter the blood circuit and the circuit's blood level drops, this blood level drop would occur in the bubble trap rather than in the blood mixing chamber. As a result, the delivery system is capable of re-priming the blood circuit without shutting off and thus without requiring the therapy to be stopped and restarted. Instead, the therapy may be delayed or paused in order for the system to be re-primed, which removes the air bubble from the blood circuit. Re-priming or removal of air bubbles can be performed while the system remains connected to the patient as previously described. The air bubbles can be vented from the bubble trap, and the bubble trap refilled with blood to the proper level. The automatic bidirectional priming capability of the delivery system can facilitate recovery of the system after detection of an air bubble in the blood circuit, during therapy delivery, without requiring system shut down or reboot.

FIG. 2B is a diagram of an example of a portion of the system 100 of FIG. 1 including a cartridge 260. The cartridge 260 of FIG. 2B is similar to the cartridge 200 of FIG. 2A, except that the bubble trap 208, draw pressure sensor 240, bubble trap solenoid 204, and bubble trap vent 234 are removed. The cartridge 260 enables the controller 102 to prime the system 100 with a one-touch priming process that is different than the process described in relation to FIG. 2A. The cartridge 260 has a draw line 214 that is connected directly to the pump 210. The priming process for the cartridge 260 uses air in the tubing (e.g., the draw line 214 and/or the return line 218) to assist in the priming process. In some aspects, a draw line 214 pressure sensor can be optionally included.

The controller 102 initiates a self-test in which the system 100 tests the cartridge 260, flow control mechanisms 216 and 212 (e.g., a return line clamp and a draw line clamp), a valve or vent 232 in the blood mixing chamber 206, the pump 210, and the pressure sensor 238. These sensors and devices are tested for correct operation and responsiveness to control signals from the controller 102. The system 100 determines automatically whether a sensor or hardware device has failed to ensure that no air is advanced to the patient. Once the priming sequence is completed, the controller 102 also determines whether any air is remaining in the draw line 214 or the return line 218, as previously described, to ensure that the priming process is successful and to ensure that no air is advanced to the patient.

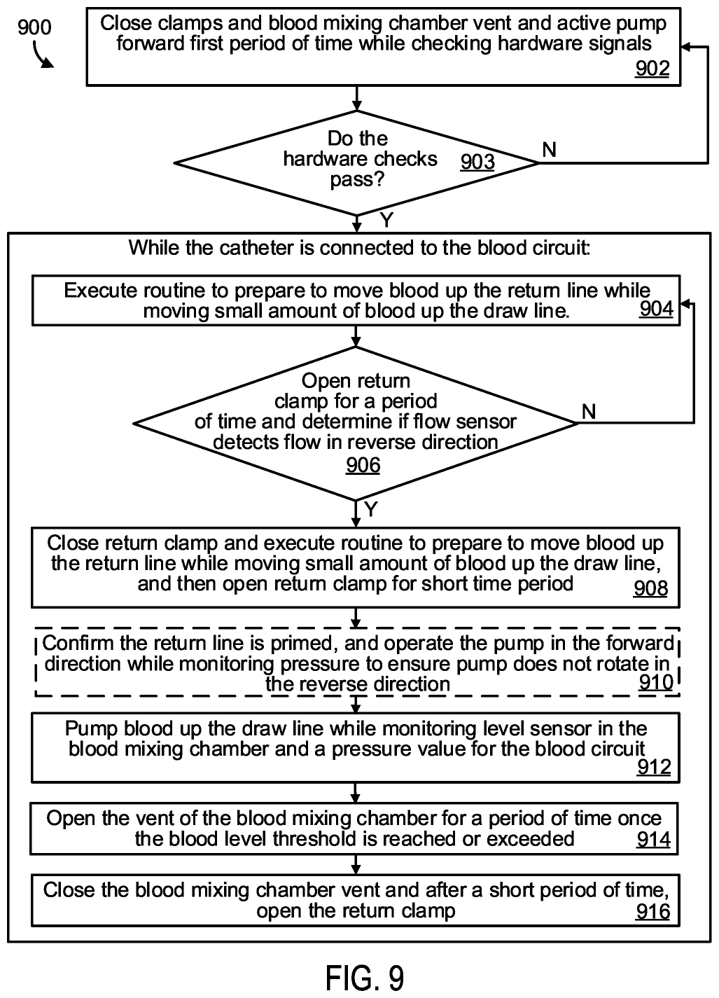

Once the hardware devices in the fluid loop are tested, the controller 102 initiates the priming process. The system 100 uses air in the blood circuit to perform the priming sequence. The controller 102 causes the pump 210 to operate to compress air in the blood mixing chamber 206. The vent 230 is then opened to force the air from the blood circuit. The pump 210 is capable of pumping air and fluid in either direction in the blood circuit. The pump 210 operates to force air out of the blood mixing chamber 206 during the priming sequence. The return line flow control mechanism 216 closes to ensure that no air that is pumped from the draw line 212 is pumped through the return line 216 to the patient. The air is compressed in the blood mixing chamber 206 and vented by vent 230. The priming process 900 related to the hardware of FIG. 2B is described in detail with respect to FIG. 9.

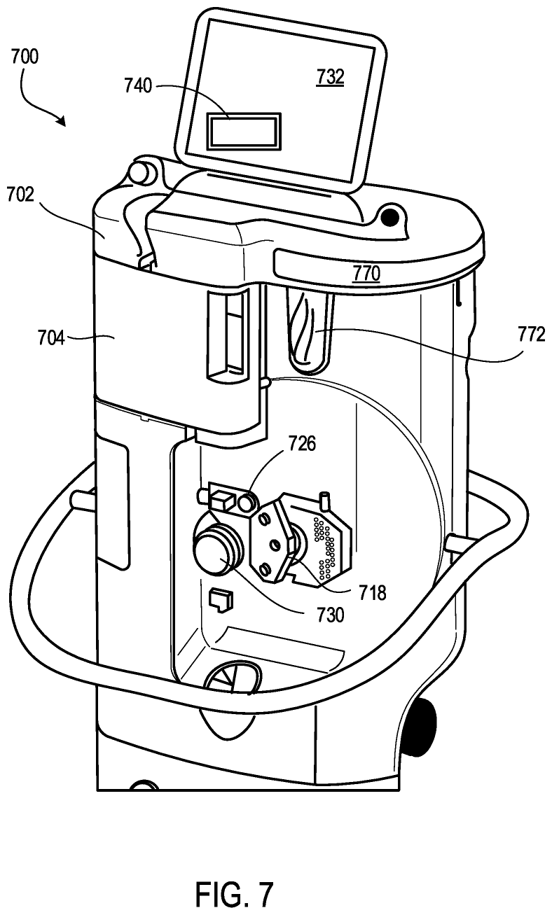

FIG. 7 is an example of a delivery system 700 such as the delivery system 100 of FIGS. 1-6. The delivery system 700 includes a console housing 702 which supports the components of the blood circuit. The delivery system 700 includes a first flow control mechanism in the form of a return clamp 730, and a second flow control mechanism in the form of a draw clamp (not shown), a pump 718, and a flow probe 726. A housing door 704 is configured to shut over a cartridge (not shown), which may include blood mixing chamber 764 of the cartridge, which sits in the cartridge housing behind the cartridge door. A priming actuator 740 is shown on a user interface 732. A fluid source provides saline to the oxygenator of the cartridge (not shown). A power lever 772 for activating the delivery system 700 is shown in an ON position.

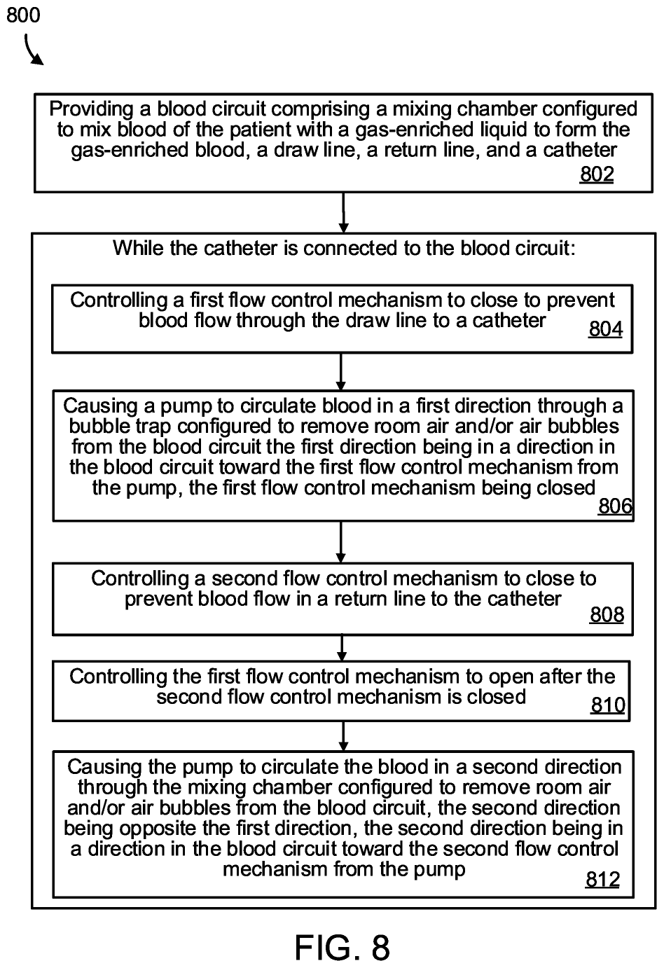

FIG. 8 shows a flow diagram of an example process 800 for automatic bidirectional priming of a delivery system for delivering gas-enriched blood within the vasculature of a patient (e.g., delivery system 100 of FIGS. 1-6 and delivery system 700 of FIG. 7). The process 800 is for automated bidirectional priming of a blood circuit while a catheter is connected to the blood circuit. The blood circuit is configured for delivering gas-enriched blood to a vasculature of a patient, as previously described. The process 800 includes providing (802) a blood circuit comprising a mixing chamber configured to mix blood of the patient with a gas-enriched liquid to form the gas-enriched blood, a draw line, a return line, and a catheter. The draw line and return line are connected to the catheter. While the catheter is connected to the blood circuit, the following steps of the process 800 are performed. A priming actuator may be pressed and released. The system is configured to close a section of the draw line by a first flow control mechanism (e.g., a clamp, valve, etc.) to prevent blood flow through the draw line to a catheter e.g., by controlling a flow control mechanism to close or block the section of the draw line. The process 800 includes causing (806) a pump to circulate blood in a first direction through the mixing chamber and through a bubble trap configured to remove room air and/or air bubbles from the blood circuit, the first direction being in a direction in the blood circuit toward the closed draw line from the pump. The process 800 includes closing a section of the return line to prevent blood flow in a return line to the catheter e.g., by controlling a second flow control mechanism (e.g., a clamp or valve) to close. The process 800 includes opening the draw line after the return line is closed. The process 800 includes causing (812) the pump to circulate the blood in a second direction through the mixing chamber configured to remove room air and/or air bubbles from the blood circuit. The second direction is opposite the first direction in the blood circuit. The second direction is in a direction in the blood circuit toward the closed return line from the pump. Optionally, the sequence above may be reversed where the system is configured to close the return line to a catheter first and then close the draw line to the catheter.

FIG. 9 shows a flow diagram including an example process 900 for automatically priming the system of FIG. 2B for delivering gas-enriched blood within the vasculature of a patient. The priming sequence 900 includes the following actions. The controller of the system (e.g., controller 102 of system 100) is configured to close (902) any flow control mechanisms (e.g., clamps) on the draw and return lines and test the blood mixing chamber vent actuation. The controller activates the pump forward for a first period of time while checking signals from each of these devices. The controller determines (903) whether the hardware tests are passed by the hardware devices. The devices pass if they actuate correctly and provide data indicative of correct actuation (e.g., opening and closing responsive to respective signaling). If the controller determines that a test is failed, the priming sequence stops and requests intervention or restarts the tests until they are passed. The controller 102 begins the priming process, which can be conducted while the catheter is connected to the blood circuit. The controller executes (904) a routine to prepare to move blood up the return line while moving a small amount of blood up the draw line. This includes activating the pump in both a forward and a reverse direction, as subsequently described in detail. The controller is configured to open return flow control mechanism (e.g., a clamp) for a period of time (a few seconds) and determine (906) if the flow sensor detects blood flow in a reverse direction. The reverse direction includes blood flowing toward the draw line from the return line.

The controller is configured to close (908) the return flow control mechanism (e.g., a return clamp) and execute a pumping routine to prepare to move blood up the return line while moving small amount of blood up the draw line. The controller then opens the return control mechanism for a short period of time (about 2 seconds).

The controller is configured to confirm (910) that the return line is primed, or filled with blood. The controller determines there is a small amount of blood (about 0.25 inches depth) in the blood mixing chamber. The controller only operates the pump in a forward direction, subsequently. The controller monitors the pressure in the blood circuit to confirm that the pump does not rotate or has not rotated in a reverse direction. The step 910 in which the controller actively checks the status of the system can be optional. In some implementations, the return line is primed and there is about 0.25 inches of blood in the blood mixing chamber. The pump operates in a forward direction after the return line is primed. The controller monitors/measures the pressure in the blood circuit to ensure that the pump is not rotating in the reverse direction.

The process 900 includes pumping (912) the blood up the draw line while monitoring level sensor in the blood mixing chamber and a pressure value for the blood circuit. The controller 102 opens (914) the vent of the blood mixing chamber for a period of time once the blood level threshold is reached or exceeded. The controller closes (916) the blood mixing chamber vent and after a short period of time, opens the return clamp. This completes the priming sequence.

A particular embodiment of the priming sequence 900 is now described in greater detail. The priming sequence 900 can use the cartridge 260 of FIG. 2B. The pump can operate up to 60 rotations per minute. The controller closes a return line clamp and a draw line clamp. The blood mixing chamber vent is closed. The controller activates the pump in a forward direction for a period of time. In an aspect, the period of time is up to 8 seconds or if more than 80 mmHg pressure is detected by the controller through the pressure sensor. The controller stops the pump and monitors pressure over a period of time. The controller determines whether the pressure value remains static. If the pressure does not reach a threshold (such as 40 mmHg), or if the pressure drops more than a threshold amount (such as 5 mmHg), the controller determines that the return clamp or the chamber vent is not closing and the priming process stops. If the controller detects a pressure above a threshold value (such as 80 mmHg), the controller determines that the draw line clamp is not closing and stops the priming process.

The controller opens the draw line clamp for a period of time (such as about 2-5 seconds). Blood is drawn up the draw line. The controller activates the pump to pump forward for a period of time (up to 10 seconds) or until the pressure stabilizes near a threshold value (such as 140 mmHg). The controller opens the draw line clamp for a short period of time (about 2-5 seconds) and closes the draw line clamp after this period of time expires. Blood is drawn up the draw line.

The controller activates the pump in a forward direction for a period of time (about 5-15 seconds). The pressure is monitored by the controller until the pressure value stabilizes. The pressure is checked against a target value (at least 200 mmHg). If the pressure is above the target value, the controller opens the chamber vent. If the pressure is not above the target value, the controller opens the draw line clamp for a short period of time (2-5 seconds) and repeats operation of the pump in the forward direction for the period of time (5-15 seconds), and checks for the pressure for the target value. The controller repeats this step once; after this step is repeated, the controller opens the blood mixing chamber vent, regardless of whether the pressure is above the target value.

The pressure in the blood mixing chamber is reduced to near zero or zero. The controller then closes the chamber vent. There is vacuum between draw clamp and the pump. The vacuum is a lower pressure than the ambient pressure, and is also called a negative pressure. The controller is configured to measure this negative or vacuum pressure. The controller activates the pump in reverse for a period of time (e.g., 11 seconds, or between 5-15 seconds). The pump creates a pressure of about 30 pounds per square inch (PSI) pressure between the pump and draw clamp. 1 PSI is 51.7149 mmHg-28″ water. A vacuum is generated in the blood mixing chamber.

The controller opens the return clamp for a short period of time (2-5 seconds, or about 3 seconds) or until flow is detected. The controller then closes the return clamp.

The controller activates the pump in a forward direction for a period of time (5-15 seconds, or about 10 seconds) or until the pressure rises and stabilizes near a target value (such as about 60 mmHg). The controller opens the draw clamp for a short period of time (2-5 seconds, or about 3 seconds) and then closes the draw clamp. Blood is drawn into the draw line.

The controller activates the pump in a forward direction for a period of time (5-15 seconds or about 10 seconds) or until pressure rises and stabilizes near a target value (such as about 140 mmHg). The controller opens the draw clamp for a short period of time (2-5 seconds or about 3 seconds) and closes the draw clamp. Blood is drawn up the draw line.

The controller activates the pump in a forward direction for a period of time (5-15 seconds or about 10 seconds). The controller monitors the pressure that increases until it stabilizes near a target value (such as at least 200 mmHg). The controller checks the pressure value against the target value. If the pressure is above the pressure, the controller opens the blood mixing chamber vent. If the pressure is not above the target value, the controller opens the draw line clamp for a short period of time (2-5 seconds, or about 3 seconds) and repeats the previous step. The controller repeats this step once; after that, the controller opens the blood mixing chamber vent, regardless of the pressure value. The pressure is reduced to about zero. The controller then closes the blood mixing chamber vent, and there is a vacuum between the draw clamp and the pump.

The controller activates the pump in a reverse direction (to pump towards the draw clamp) for a period of time (5-15 seconds, or about 11 seconds). The pump creates up to about 30 PSI of pressure between the pump and draw clamp and a vacuum in the blood mixing chamber.

The controller opens the return clamp until a blood flow is detected. The controller then closes the return clamp a short period (about 2 seconds) after blood flow is detected. The controller causes the fluid to raise in the blood mixing chamber (generally about a quarter inch) up from the bottom of the chamber. The priming of the return line is now completed.

The controller activates the pump to pump forward for a short period of time (about 6 seconds). The pressure increases and stabilizes. The controller checks to determine that the pressure is near a target value (about 80 mmHg). The controller opens the draw clamp. Blood moves up the draw line, and pressure equalizes with the blood pressure of the patient.

The controller activates the pump in a forward direction to pump blood toward the return line. When the pressure reaches a threshold value (about 200 mmHg), the controller opens the blood mixing chamber vent for a short period of time (about 2 seconds). When the lower level sensor senses the blood level, the controller opens the blood mixing chamber vent for a period of time (about 3 seconds) regardless of the pressure. The controller then closes the blood mixing chamber vent. The controller stops the pump after a period of time (at least 2 seconds) or when pressure in blood mixing chamber reaches a maximum allowed pressure (about 1900 mmHg). The draw clamp is opened and the return clamp is closed. The priming process is completed.

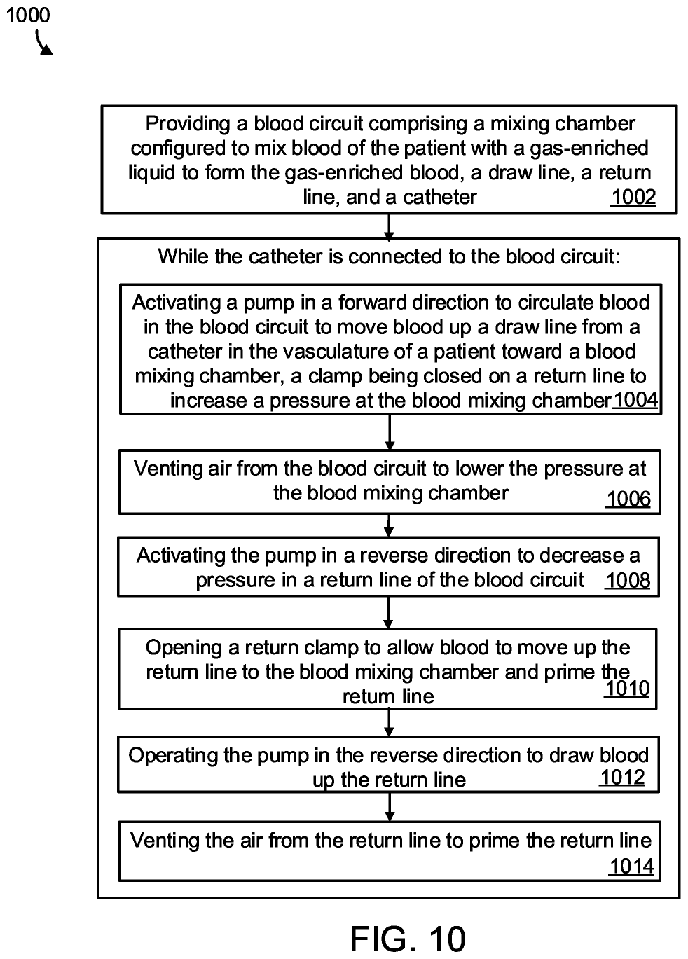

FIG. 10 shows a flow diagram including an example process 1000 for automatically priming the system of FIG. 1 and FIG. 2B for delivering gas-enriched blood within the vasculature of a patient. The process 1000 includes providing (1002) a blood circuit comprising a mixing chamber configured to mix blood of the patient with a gas-enriched liquid to form the gas-enriched blood, a draw line, a return line, and a catheter. The process 1000 includes, while the catheter is connected to the blood circuit, performing the following operations. The process 1000 includes activating (1004) a pump in a forward direction to circulate blood in the blood circuit to move blood up a draw line from a catheter in the vasculature of a patient toward a blood mixing chamber, a clamp being closed on a return line to increase a pressure at a blood mixing chamber. The process 1000 includes venting (1006) air from the blood circuit to lower the pressure at the blood mixing chamber. The process 1000 includes activating (1008) the pump in a reverse direction to decrease a pressure in a return line of the blood circuit. The process 1000 includes opening (1010) a return clamp to draw blood up the return line to the blood mixing chamber and prime the return line by drawing blood up the return line (in a reverse direction) to prime the return line. The process 1000 includes activating (1012) the pump in the reverse direction to draw blood up the return line. The process 1000 includes venting (1014) the air from the return line to prime the return line.

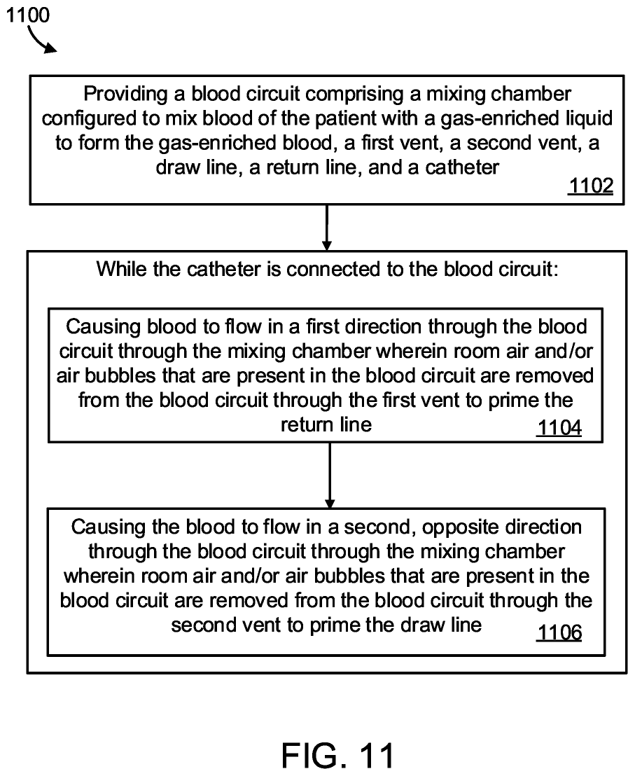

FIG. 11 shows a flow diagram including an example process 1100 for automatically priming the system of FIG. 1 for delivering gas-enriched blood within the vasculature of a patient. The process 1100 is for automated bidirectional priming of a blood circuit while a catheter is connected to the blood circuit, the blood circuit configured for delivering gas-enriched blood to a vasculature of a patient. The process 1100 comprises providing (1102) a blood circuit comprising a mixing chamber configured to mix blood of the patient with a gas-enriched liquid to form the gas-enriched blood, a first vent, a second vent, a draw line, a return line, and a catheter, wherein the draw line and return line are connected to the catheter. The process 1100 includes performing the following operations while the catheter is connected to the blood circuit. The process 1100 includes causing (1104) blood to flow in a first direction through the blood circuit through the mixing chamber wherein room air and/or air bubbles that are present in the blood circuit are removed from the blood circuit through the first vent to prime the return line. The process 1100 includes causing (1106) the blood to flow in a second, opposite direction through the blood circuit through the mixing chamber wherein room air and/or air bubbles that are present in the blood circuit are removed from the blood circuit through the second vent to prime the draw line.

In some implementations, the process 1100 includes closing a first flow control mechanism when causing the blood to flow in the first direction, the first flow control mechanism blocking blood flow in the draw line. In some implementations, the process 1100 includes closing a second flow control mechanism and opening the first flow control mechanism when causing the blood to flow in the second direction, the second flow control mechanism blocking blood flow in the return line. In some implementations, the process 1100 includes measuring, by a first pressure sensor, a first pressure in the blood circuit between a pump and a first flow control mechanism in the blood circuit when the pump is causing the blood to flow in the first direction through the blood circuit. The process 1100 includes comparing the first pressure to a threshold value. The process 1100 includes determining that the return line is primed when the first pressure exceeds the threshold value.

In some implementations, the process 1100 includes measuring, by a second pressure sensor, a second pressure in the blood circuit between a pump and a second flow control mechanism in the blood circuit when the pump is causing the blood to flow in the second direction through the blood circuit. The process 1100 includes comparing the second pressure to a threshold value. The process 1100 includes determining that the draw line is primed when the first pressure exceeds the threshold value.

In some implementations, the process 1100 includes determining that the draw line is primed, determining that the return line is primed, and in response to determining each of the draw line and the return line are primed, causing circulation of blood through a catheter coupled to the draw line and the return line.

In some implementations, the process 1100 includes receiving sensor data from one or more of a pressure sensor, blood level sensor, a pump, a first flow control mechanism, and a second flow control mechanism. The process 1100 includes determining, based on the sensor data, that a priming process is successful or unsuccessful.

In some implementations, determining, based on the sensor data, that the priming process is successful or unsuccessful includes comparing pressure data to a threshold; and in response to determining that the pressure data satisfies the threshold, determining that the priming process is successful. In some implementations, determining, based on the sensor data, that the priming process is successful or unsuccessful comprises receiving, from a first blood level sensor in the mixing chamber, first blood level data indicating that the mixing chamber is full of blood; receiving, from a second blood level sensor in a bubble trap second blood level data indicating that the bubble trap is full of blood; and in response to receiving the first blood level data and the second blood level data, determining that the priming process is successful.

In some implementations, the process 1100 includes actuating a control; and in response to actuation of the control, automatically causing the blood to flow in the first direction through the blood circuit through the mixing chamber to prime the return line and automatically causing the blood to flow in the second, opposite direction through the blood circuit to prime the draw line.

In some implementations, the process 1100 includes generating a data log comprising operational data that describes the automated bidirectional priming of the blood circuit.

In some implementations, the process 1100 includes detecting that the automated bidirectional priming of the blood circuit is completed; and in response to detecting, sending the data log to a remote storage comprising cloud storage.

In some implementations, the process includes receiving a query for data describing operation of a pump, a pressure sensor, a temperature sensor, a first flow control mechanism on the draw line, or a second flow control mechanism on the return line; and in response to receiving the query, sending at least a portion of the data log to a remote device.

In some implementations, the process 1100 includes determining that a value included in the data log is outside an expected range for that value; and generating data indicating that an error occurred during the automated bidirectional priming of the blood circuit.

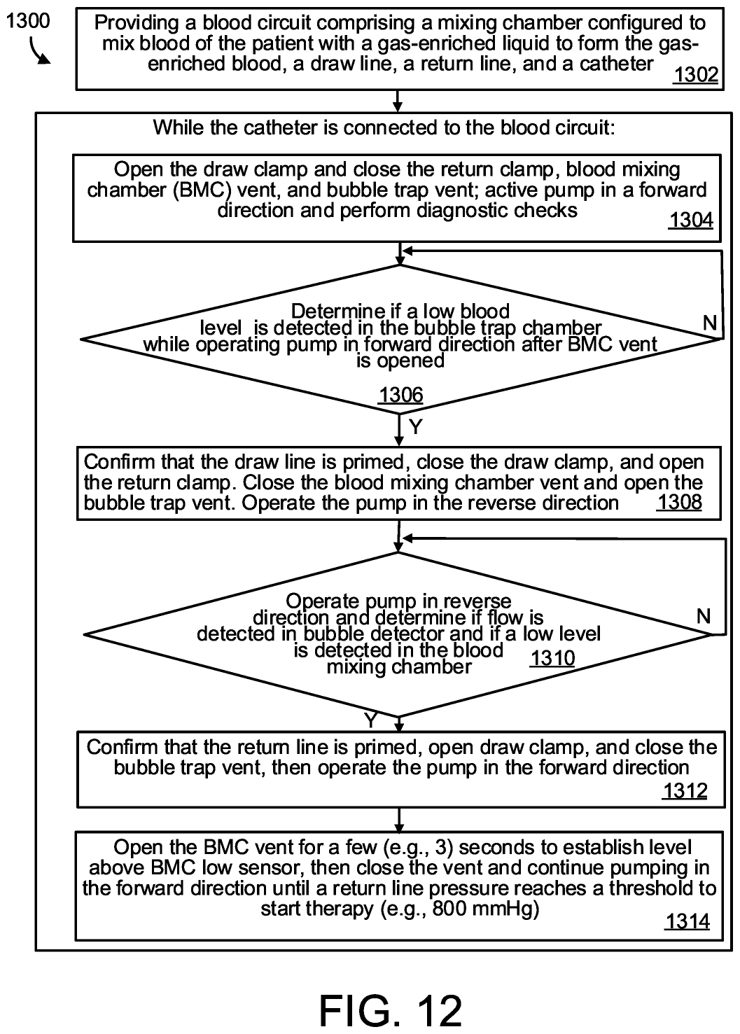

FIG. 12 shows a flow diagram including an example process 1300 for automatically priming the system with cartridge 200 of FIG. 2A for delivering gas-enriched blood within the vasculature of a patient. The cartridge 200 includes a bubble trap and bubble trap vent. In some implementations, process 1300 is less timing dependent than the process 900 that does not include a bubble trap (e.g., related to cartridge 260 of FIG. 2B).

Generally, the priming process 1300 can be initiated by a user. In an example, after the user has loaded the cartridge 200, prepped the cartridge 200, and made patient connections as described previously and on the console user interface, the user initiates prime by pressing a button on the user interface of the system 100.

Generally, when the priming process 1300 begins, all vents (e.g., vents 230 and 234) and clamps (e.g., mechanisms 212 and 216) are closed. The controller causes the draw clamp to open and operates the pump in a forward direction. The controller performs pressure checks to ensure that no leaks are occurring in the clamps or tubing. The checks verify that the cartridge and tube set is properly loaded by monitoring for a pressure increase in the return pressure sensor.

When the pressure checks are verified, the blood mixing chamber vent (e.g., vent 230) is opened. The controller continues priming the draw line until a blood level is detected by a bubble trap low sensor (e.g., sensor 602). When this blood level is detected, the draw line is primed.

The controller closes the draw line clamp. The controller operates the pump in a reverse direction. The controller opens the return line clamp. The controller closes the blood mixing chamber vent (e.g., vent 230) and opens the bubble trap vent (e.g., vent 234). When the controller detects the flow probe signal, and the controller detects a blood mixing chamber low level, the return line is primed.

The controller operates the pump in a forward direction. The controller opens the draw clamp and closes the bubble trap vent. The controller opens the blood mixing chamber vent momentarily to establish a blood level above the low level sensor. The controller then closes the blood mixing chamber vent. The priming sequence is complete when the pressure sensor detects minimum treatment pressure (e.g., at least 800 mmHg).

The priming sequence 1300 includes the following actions. The process 1300 includes providing (1302) a blood circuit comprising a mixing chamber configured to mix blood of the patient with a gas-enriched liquid to form the gas-enriched blood, a draw line, a return line, and a catheter. The process 1300 includes, while the catheter is connected to the blood circuit, performing the following operations. The process 1300 includes activating (1004) a pump in a forward direction to circulate blood in the blood circuit to move blood up a draw line from a catheter in the vasculature of a patient toward a blood mixing chamber, a clamp being closed on a return line to increase a pressure at a blood mixing chamber. The clamp on the draw line is open. The vent on the blood mixing chamber is closed. The vent on the bubble trap is closed. Diagnostic checks are performed, which include pressure checks previously described. If these checks are passed, the processed continues. Else, the process stops and solicits further user input.

The process 1000 includes determining (1306) if a flow sensor detects fluid flow in the blood circuit in a reverse direction. The controller determines (1306) if a low blood level is detected in the bubble trap while operating pump in forward direction after the bubble trap vent is opened by the controller. If a low blood level sensor is triggered, the controller proceeds to step 1308. Else, the controller continues operating the pump in the forward direction as in step 1306.

The controller confirms (1308) that the draw line is primed. The controller closes the draw clamp and opens the return clamp. The controller closes the blood mixing chamber vent and opens the bubble trap vent. The controller operates the pump in the reverse direction.

The controller is configured to continue operating (1310) the pump in the reverse direction and determine if a fluid flow is detected in bubble detector and if a low level is detected in the blood mixing chamber. If these criteria are not satisfied, the controller continues to operate the pump in reverse as in step 1310. When these criteria are satisfied, the return line is primed.

The controller confirms (1312) that the return line is primed. The controller opens the draw clamp and closes the bubble trap vent. The controller operates the pump in the forward direction.

The controller opens (1314) the blood mixing chamber vent for a few (e.g., 3-5) seconds to establish a blood level above a blood mixing chamber low sensor. The controller then closes the blood mixing chamber vent and continues operating the pump in the forward direction until a return line pressure reaches a threshold (about 800 mmHg) to start therapy. The bi-direction priming sequence is then complete.

Some implementations of subject matter and operations described in this specification (e.g., processes 800, 900, 1000, 1100, and 1300) can be implemented in digital electronic circuitry, or in computer software, firmware, or hardware, including the structures disclosed in this specification and their structural equivalents, or in combinations of one or more of them. For example, in some implementations, the processor of the delivery system (e.g., delivery system 100) can be implemented using digital electronic circuitry, or in computer software, firmware, or hardware, or in combinations of one or more of them.

Some implementations described in this specification (e.g., the processor of the delivery system, etc.) can be implemented as one or more groups or modules of digital electronic circuitry, computer software, firmware, or hardware, or in combinations of one or more of them. Although different modules can be used, each module need not be distinct, and multiple modules can be implemented on the same digital electronic circuitry, computer software, firmware, or hardware, or combination thereof.

Some implementations described in this specification can be implemented as one or more computer programs, i.e., one or more modules of computer program instructions, encoded on computer storage medium for execution by, or to control the operation of, data processing apparatus. A computer storage medium can be, or can be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or be included in, one or more separate physical components or media (e.g., multiple CDs, disks, or other storage devices).

The term “data processing apparatus” encompasses all kinds of apparatus, devices, and machines for processing data, including by way of example a programmable processor, a computer, a system on a chip, or multiple ones, or combinations, of the foregoing. The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing, and grid computing infrastructures.

A computer program (also known as a program, software, software application, script, or code) can be written in any form of programming language, including compiled or interpreted languages, declarative or procedural languages. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed for execution on one computer or on multiple computers that are located at one site or distributed across multiple sites and interconnected by a communication network.

Some of the processes and logic flows described in this specification can be performed by one or more programmable processors executing one or more computer programs to perform actions by operating on input data and generating output. The processes and logic flows can also be performed by, and apparatus can be implemented as, special purpose logic circuitry, e.g., an FPGA (field programmable gate array) or an ASIC (application specific integrated circuit).

Processors suitable for the execution of a computer program include, by way of example, both general and special purpose microprocessors, and processors of any kind of digital computer. Generally, a processor will receive instructions and data from a read only memory or a random-access memory or both. A computer includes a processor for performing actions in accordance with instructions and one or more memory devices for storing instructions and data. A computer may also include, or be operatively coupled to receive data from or transfer data to, or both, one or more mass storage devices for storing data, e.g., magnetic, magneto optical disks, or optical disks. However, a computer need not have such devices. Devices suitable for storing computer program instructions and data include all forms of non-volatile memory, media and memory devices, including by way of example semiconductor memory devices (e.g., EPROM, EEPROM, flash memory devices, and others), magnetic disks (e.g., internal hard disks, removable disks, and others), magneto optical disks, and CD-ROM and DVD-ROM disks. The processor and the memory can be supplemented by, or incorporated in, special purpose logic circuitry.

To provide for interaction with a user, operations can be implemented on a computer having a display device (e.g., a monitor, or another type of display device) for displaying information to the user and a keyboard and a pointing device (e.g., a mouse, a trackball, a tablet, a touch sensitive screen, or another type of pointing device) by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

A computer system may include a single computing device, or multiple computers that operate in proximity or generally remote from each other and typically interact through a communication network. Examples of communication networks include a local area network (“LAN”) and a wide area network (“WAN”), an inter-network (e.g., the Internet), a network comprising a satellite link, and peer-to-peer networks (e.g., ad hoc peer-to-peer networks). A relationship of client and server may arise by virtue of computer programs running on the respective computers and having a client-server relationship to each other.

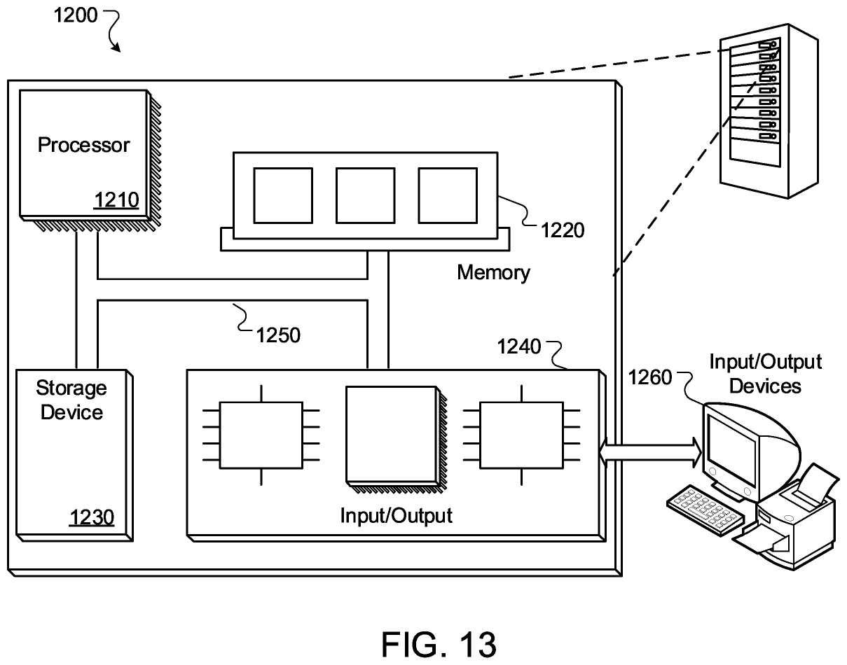

FIG. 13 shows an example computer system 1200 that includes a processor 12100, a memory 1220, a storage device 1230 and an input/output device 1240. Each of the components 12100, 1220, 1230 and 1240 can be interconnected, for example, by a system bus 1250. The processor 12100 is capable of processing instructions for execution within the system 1200. In some implementations, the processor 12100 is a single-threaded processor, a multi-threaded processor, or another type of processor. The processor 12100 is capable of processing instructions stored in the memory 1220 or on the storage device 1230. The memory 1220 and the storage device 1230 can store information within the system 1200.

The input/output device 1240 provides input/output operations for the system 1200. In some implementations, the input/output device 1240 can include one or more of a network interface device, e.g., an Ethernet card, a serial communication device, e.g., an RS-232 port, and/or a wireless interface device, e.g., an 802.11 card, a 3G wireless modem, a 4G wireless modem, a 5G wireless modem, etc. In some implementations, the input/output device can include driver devices configured to receive input data and send output data to other input/output devices, e.g., keyboard, printer and display devices 1260. In some implementations, mobile computing devices, mobile communication devices, and other devices can be used.

While this specification contains many details, these should not be construed as limitations on the scope of what may be claimed, but rather as descriptions of features specific to particular examples. Certain features that are described in this specification in the context of separate implementations can also be combined. Conversely, various features that are described in the context of a single implementation can also be implemented in multiple embodiments separately or in any suitable sub-combination.

A number of embodiments have been described. For example, the detailed description and the accompanying drawings to which it refers are intended to describe some, but not necessarily all, examples or embodiments of the system. The described embodiments are to be considered in all respects only as illustrative and not restrictive. Nevertheless, various modifications may be made without departing from the scope of the data processing system described herein. Accordingly, other embodiments are within the scope of the following claims.

1. A delivery system for delivering gas-enriched blood within a vasculature of a patient, the delivery system configured for automated priming of a blood circuit of the delivery system, the delivery system comprising:
a blood circuit, comprising:
a pump configured to circulate blood in the blood circuit;
a mixing chamber configured to mix blood of the patient with a gas-enriched liquid to form a gas-enriched blood;
a draw line coupled to the mixing chamber and configured to connect a catheter to the mixing chamber and to interface with a first flow control mechanism;
a return line coupled to the mixing chamber and configured to connect the catheter to the mixing chamber and to interface with a second flow control mechanism; and
a controller configured to control operation of the pump and operation of the first and second flow control mechanisms to perform bidirectional priming of the blood circuit while the catheter is connected to the blood circuit, the controller configured for alternating a direction of blood flow through the blood circuit and alternating closure of the first and second flow control mechanisms to block blood flow in the draw line and return line and prevent room air and/or air bubbles from flowing to the catheter during priming.

2. The delivery system of claim 1, wherein the controller is configured to perform operations comprising:
closing the second flow control mechanism when causing the blood to flow in forward direction, the second flow control mechanism blocking blood flow in the return line.

3. The delivery system of claim 2, wherein the controller is configured to perform operations comprising:
closing the first flow control mechanism and opening the second flow control mechanism when causing the blood to flow in a reverse direction, the first flow control mechanism blocking blood flow in the draw line.

4. The delivery system of claim 1, wherein the controller is configured to perform operations comprising:
measuring, by a first pressure sensor, a first pressure in the blood circuit between the pump and the second flow control mechanism in the blood circuit when the pump is causing the blood to flow in a first direction through the blood circuit;
comparing the first pressure to a threshold value; and
determining that the return line is primed when the first pressure exceeds the threshold value.

5. The delivery system of claim 4, wherein the controller is configured to perform operations comprising:
measuring, by a second pressure sensor, a second pressure in the blood circuit between a pump and a first flow control mechanism in the blood circuit when the pump is causing the blood to flow in a second direction through the blood circuit;
comparing the second pressure to a threshold value; and
determining that the draw line is primed when the second pressure exceeds the threshold value.

6. The delivery system of claim 1, wherein the controller is configured to perform operations comprising:
determining that the draw line is primed;
determining that the return line is primed; and
in response to determining each of the draw line and the return line are primed, causing circulation of blood through the catheter coupled to the draw line and the return line.

7. The delivery system of claim 1, wherein the controller is configured to perform operations comprising:
receiving sensor data from one or more of a pressure sensor, blood level sensor, the pump, the first flow control mechanism, and the second flow control mechanism; and
determining, based on the sensor data, that a priming process is successful or unsuccessful.

8. The delivery system of claim 7, wherein determining, based on the sensor data, that the priming process is successful or unsuccessful comprises:
comparing pressure data to a threshold; and
in response to determining that the pressure data satisfies the threshold, determining that the priming process is successful.

9. The delivery system of claim 7, wherein determining, based on the sensor data, that the priming process is successful or unsuccessful comprises:
receiving, from a first blood level sensor in the mixing chamber, first blood level data indicating that the mixing chamber is full of blood;
receiving, from a second blood level sensor in a bubble trap second blood level data indicating that the bubble trap is full of blood; and
in response to receiving the first blood level data and the second blood level data, determining that the priming process is successful.

10. The delivery system of claim 1, wherein the controller is configured to perform operations comprising:
determining that a control is actuated; and
in response to determining, causing the blood to flow in a first direction through the blood circuit through the mixing chamber to prime the return line and causing the blood to flow in a second, opposite direction through the blood circuit to prime the draw line.

11. The delivery system of claim 1, wherein the controller is configured to perform operations comprising:
generating a data log comprising operational data that describes a bidirectional priming of the blood circuit.

12. The delivery system of claim 11, wherein the controller is configured to perform operations comprising:
detecting that the bidirectional priming of the blood circuit is completed; and
in response to detecting, sending the data log to a remote storage comprising cloud storage.

13. The delivery system of claim 11, wherein the controller is configured to perform operations comprising:
receiving a query for data describing operation of a pump, a pressure sensor, a temperature sensor, the first flow control mechanism on the draw line, or the second flow control mechanism on the return line; and
in response to receiving the query, sending at least a portion of the data log to a remote device.

14. The delivery system of claim 11, wherein the controller is configured to perform operations comprising:
determining that a value included in the data log is outside an expected range for that value; and
generating data indicating that an error occurred during the bidirectional priming of the blood circuit.

15. The delivery system of claim 1, wherein the gas-enriched blood is formed by mixing the blood with oxygen-enriched liquid having a dissolved O₂ concentration of 0.1-6 ml O₂/ml liquid.

16. The delivery system of claim 1, wherein the gas-enriched blood is oxygen-enriched blood having an elevated pO₂ of 600-1500 mmHg.

17-81. (canceled)

82. A method for priming of a blood circuit of a gas-enriched blood system, the method comprising:
performing, by a controller, a bidirectional priming of the blood circuit while a catheter is connected to the blood circuit, the controller configured for alternating a direction of blood flow through the blood circuit and alternating closure of first and second flow control mechanisms to alternatively block blood flow in a draw line a return line and prevent room air and/or air bubbles from flowing to the catheter during priming.

83-118. (canceled)

119. The method of claim 82, wherein the controller is configured to perform operations comprising:
closing the second flow control mechanism when causing the blood to flow in forward direction, the second flow control mechanism blocking blood flow in the return line.

120. The method of claim 119, wherein the controller is configured to perform operations comprising:
closing the first flow control mechanism and opening the second flow control mechanism when causing the blood to flow in a reverse direction, the first flow control mechanism blocking blood flow in the draw line.

121. The method of claim 82, wherein the controller is configured to perform operations comprising:
receiving sensor data from one or more of a pressure sensor, blood level sensor, a pump, the first flow control mechanism, or the second flow control mechanism; and
determining, based on the sensor data, that a priming process is successful or unsuccessful.
